(19) United States
(12) Patent Application Publication
(10) Pub. No.: US 2026/0224901 A1
(43) Pub. Date: Aug. 6, 2026

(54) DETERMINATION OF SEPTAL PERFORATION DURING ELECTRODE IMPLANTATION

(71) Applicant: Medtronic, Inc., Minneapolis, MN (US)

(72) Inventors: Xiaohong Zhou, Woodbury, MN (US); Jian Cao, San Jose, CA (US); Wade M. Demmer, Coon Rapids, MN (US)

(21) Appl. No.: 19/149,321

(22) PCT Filed: Jan. 8, 2024
(86) PCT No.: PCT/IB2024/050161

§ 371 Date: Jul. 18, 2025

Related U.S. Application Data
(60) Provisional application No. 63/441,684, filed on Jan. 27, 2023.

(51) Int. Cl. A61N 1/37; A61N 1/05

(52) U.S. Cl. CPC A61N 1/3702; A61N 1/056; A61N 1/371

(57) ABSTRACT

The present disclosure relates to a system for use in assisting implantation of an implantable electrode. The system may include an implantable electrode configured to deliver cardiac conduction system pacing, and an external electrode configured to sense electrical activity. The system may further include a computing apparatus operably coupled to the implantable electrode and external electrode, and configured to monitor at least one of internal and external electrical activity and determine cardiac conduction system capture, injury of current, and impedance based on the monitored electrical activity. The computing apparatus may be further configured to determine that the implantable electrode is implanted in the interventricular septal wall proximate the cardiac conduction system and prior to perforation into the left ventricular chamber based on at least one of the cardiac conduction system capture, the injury of current, and the impedance, and issue a first notification.

This application claims the benefit of U.S. Provisional Patent Application Ser. No. 63/441,684, filed Jan. 27, 2023, the entire content of which is incorporated herein by reference.

The present disclosure relates to implantable medical devices and implementation thereof, and systems and methods related thereto. In particular, the present disclosure relates to determining whether an implantable electrode is positioned within the ventricular septum of the heart of a patient near or at the cardiac conduction system based on cardiac conduction system capture, injury of current, and impedance. The present disclosure further relates to determining whether the implantable electrode has perforated the interventricular septum into the left ventricular chamber based on cardiac conduction system capture, injury of current, and impedance.

Implantable medical devices (IMDs), such as cardiac pacemakers or implantable cardioverter defibrillators, deliver therapeutic stimulation to patients' hearts thereby improving the lives of millions of patients living with heart conditions. Conventional pacing techniques involve pacing one or more of the four chambers of a patient's heart 12 as illustrated in FIG. 1, including the left atrium 33, the right atrium 26, the left ventricle 32 and the right ventricle 28. One common conventional therapeutic pacing technique that treats a slow heart rate, referred to as bradycardia, involves delivering an electrical pulse to a patient's right ventricular tissue. In response to the electrical pulse, both the right and left ventricles contract. However, the heartbeat process may be significantly delayed because the pulse travels from the right ventricle through the left ventricle. The electrical pulse passes through the muscle cells that are referred to as myocytes. Myocyte-to-myocyte conduction may be very slow. Delayed electrical pulses can cause the left ventricle to be unable to maintain synchrony with the right ventricle.

Over time, the left ventricle can become significantly inefficient at pumping blood to the body. In some patients, heart failure can develop such that the heart is too weak to pump blood to the body. Heart failure may be a devastating diagnosis since, for example, fifty percent of heart failure patients have a life expectancy of five years or less. Another possible cause of heart failure is due to dyssynchronous ventricular activation, which is an irregular or unsynchronized ventricular contraction, or due to atrioventricular dyssynchrony, which is irregular or unsynchronized time between atrial and ventricular contractions. When the ventricles beat out of sync, or when the atria of the heart beat out of sync with the ventricles of the heart, blood clots in the heart can form and increase the risk of stroke or heart failure, for example.

To avoid potential development of heart failure, some physicians have considered alternative pacing methods that involve the cardiac conduction system. Pacing the cardiac conduction system may quickly conduct electrical pulses (for example, akin to a car driving on a highway), whereas pacing cardiac muscle, or myocardial, tissue may more slowly conduct electrical pulses (for example, akin to a car driving on a dirt road).

The cardiac conduction system includes the sinoatrial node 1, atrial internodal tracts 2, 4, 5 (i.e., anterior internodal 2, middle internodal 4, and posterior internodal 5), atrioventricular node 3, His bundle 13 (also known as the atrioventricular bundle or bundle of His), left bundle branch 8a, and right bundle branch 8b as shown in FIG. 1. The arch of aorta 6 and the Bachman's bundle 7 are also shown in FIG. 1. The sinoatrial node 1, located at the junction of the superior vena cava and right atrium, is considered to be the natural pacemaker of the heart as it continuously and repeatedly emits electrical impulses. The electrical impulses spread through the muscles of right atrium 26 to left atrium 33 to cause synchronous contraction of the atria. The electrical impulses are also carried through atrial internodal tracts to the atrioventricular node 3—the sole connection between the atria and the ventricles. The conduction through the atrioventricular node or atrioventricular nodal tissue takes longer than through the atrial tissue, which results in a delay between the atrial contractions and the start of the ventricular contractions. The atrioventricular delay, which is the delay between atrial contractions and ventricular contractions, allows the atria to empty blood into the ventricles. Then, the valves between the atria and ventricles close in conjunction with ventricular contraction via branches of the bundle of His. The bundle of His, or His bundle, 13 is located in the membranous atrioventricular septum near the annulus of the tricuspid valve. The His bundle 13 splits into the left and right bundle branches 8a, 8b and are formed of specialized fibers called “Purkinje fibers” 9. The Purkinje fibers 9 may be described as being capable of rapidly conducting an action potential down the ventricular septum (VS), spreading the depolarization wavefront quickly through the remaining ventricular myocardium, and producing a coordinated contraction of the ventricular muscle mass.

Patients with a conduction system abnormality, such as poor AV node conduction or poor SA node function, may receive an IMD, such as a pacemaker, to restore a more normal heart rhythm and AV synchrony. Some types of IMDs, such as cardiac pacemakers, implantable cardioverter defibrillators (ICDs), or cardiac resynchronization therapy (CRT) devices, provide therapeutic electrical stimulation to a heart of a patient via electrodes on one or more implantable endocardial, epicardial, or coronary venous leads that are positioned in or adjacent to the heart. The therapeutic electrical stimulation may be delivered to the heart in the form of pulses or shocks for pacing, cardioversion, or defibrillation. In some cases, an IMD may sense intrinsic depolarizations of the heart, and control the delivery of therapeutic stimulation to the heart based on the sensing.

Cardiac arrhythmias may be treated by delivering electrical shock therapy for cardioverting or defibrillating the heart in addition to cardiac pacing, for example, from an ICD, which may sense a patient's heart rhythm and classify the rhythm according to an arrhythmia detection scheme in order to detect episodes of tachycardia or fibrillation.

Arrhythmias detected may include ventricular tachycardia (VT), fast ventricular tachycardia (FVT), ventricular fibrillation (VF), atrial tachycardia (AT) and atrial fibrillation (AT). Anti-tachycardia pacing (ATP), a painless therapy, can be used to treat ventricular tachycardia (VT) to substantially terminate many monomorphic fast rhythms. While ATP is painless, ATP may not deliver effective therapy for all types of VTs. For example, ATP may not be as effective for polymorphic VTs, which has variable morphologies. Polymorphic VTs and ventricular fibrillation (VFs) can be more lethal and may require expeditious treatment by shock.

Positioning an implantable electrode (e.g., on a lead, on a leadlet device, etc.) to deliver cardiac conduction system pacing as described herein requires the implantable electrode to be implanted proximate to the cardiac conduction system of the heart such as, e.g., the left bundle branch (LBB) in the ventricular septum. In some cases, during electrode implantation in the ventricular septum, the implantable electrode is advanced “too far” and the electrode or the lead (if a lead is being used) perforates the ventricular septum into a ventricular chamber, such as, e.g., the left ventricle. Such perforation may lead to loss of capture and pacing of the cardiac conduction system. Further perforation may lead to additional undesirable effects or possible lead dislodgement. Avoiding septal perforation is thus desirable in order to avoid possible negative outcomes.

### SUMMARY

This disclosure generally relates to determining whether an implantable electrode is positioned within the ventricular septum of the heart of a patient near or at the cardiac conduction system, and whether the implantable electrode should be repositioned based on that determination. The present disclosure further relates to determining that the implantable electrode has perforated the ventricular septum into the left ventricular (LV) chamber, and that the implantable electrode should be repositioned based on that determination. Determination of the position of the implantable electrode is, at least, based on cardiac conduction system capture, injury of current, and impedance. Each of cardiac conduction system capture, injury of current, and impedance may be monitored in real time, and such real-time monitoring may prevent or avoid perforation of the ventricular septum into the LV chamber. In at least one embodiment, the implantable electrode may be positioned at or near the left bundle branch for cardiac conduction system pacing.

Cardiac conduction system capture may include LBB capture, and positioning the implantable electrode near or at the cardiac conduction system may include positioning the implantable electrode near or at the LBB. Single, dual, and/or triple chamber medical devices or leadless medical devices are available that can include, for examples, a transvenous atrial lead carrying electrodes that may be placed in the right atrium, a transvenous ventricular lead carrying electrodes that may be placed in the right ventricle, or carrying electrodes that may be placed in the ventricular septum, via the right atrium, a coronary sinus lead that may be placed in the left ventricle via the coronary sinus, a ventricle-from-atrium (VfA) lead that may be placed in the right atrial septum between the right atria and the left ventricle to pace the left ventricle, and a leadless device (e.g., a leadless pacemaker for LBB pacing in the ventricular septum). During implantation of such devices, the present application may determine positioning of electrodes of such devices in the ventricular septum.

It can be difficult to implant an electrode proximate the LBB to effectively pace the LBB. Also, implanted LBB electrode(s) may dislodge over time due to natural movement or due to injury, for example, and LV septal pacing may occur as a result. This is also true for the right bundle branch (RBB) pacing shifting into right ventricular (RV) septal pacing. On one hand, for patients whose cardiac conduction systems work normally, septal pacing may be undesirable in some cases. On the other hand, for patients whose cardiac conduction systems do not work normally, septal pacing may be desirable in some cases, such as, for example, when the patient experiences LBB or RBB block which cannot be corrected or bypassed. In other cases for patients whose cardiac conduction systems do not work normally, cardiac conduction system pacing is still desirable, such as, for example, when the LBB or RBB block can be corrected or bypassed.

In particular, illustrative systems, devices and methods are described herein to determine electrode position in the ventricular septum and to determine electrode perforation through the ventricular septum into a ventricular chamber using electrogram (EGM) and electrocardiogram (ECG) signal analysis and to provide effective electrode positioning for effective pacing therapy in response thereto. Use of EGM signals, for example, may advantageously provide more efficient or more effective analysis, provide timely modifications to the pacing parameters based on a patient's changed physiological conditions resulting in more effective pacing, and may negate the need for a patient to visit a clinic to have ECG signals measured. Use of ECG signals, for example, may advantageously provide additional data from one or more surface electrodes.

Determining the position of the implantable electrode may be done using EGM and ECG signal analysis of different variables. For example, cardiac conduction system capture, injury of current, and impedance may be all be used to determine the position of the implantable electrode. Cardiac conduction system capture may be defined as successful delivery of cardiac conduction system pacing to the cardiac conduction system as opposed to delivery of the pacing, for example, to myocardial tissue. A cardiac conduction system capture threshold may be described or defined as a minimum amount of power utilized to deliver the cardiac conduction system pacing while achieving capture of the cardiac conduction system, and may be measured in volts (V). Injury of current may be described or defined as the electrical current generated when an injured part of the conduction system, muscle, or other excitable tissue is connected through a conductor with an uninjured region. The injured tissue causes a voltage difference versus the uninjured tissue. During implantation, the implantable electrode (or lead, etc.) itself may create minor tissue injury, and certain measured levels of injury of current may correspond to more optimal electrode placement. Injury of current may be measured in volts. Impedance may be described or defined as the effective resistance to pacing current arising from the combined effective of resistance and reactance in a circuit. In other words, impedance may be defined as opposition to electrical flow, and may be measured in ohms. Each of cardiac conduction system capture, injury of current, and impedance may be monitored in real time during implantation of the implantable electrode.

In alternative embodiments, not all of cardiac conduction system capture, injury of current, and impedance are monitored during implantation of the implantable electrode, and/or not all of cardiac conduction system capture, injury of current, and impedance are used to determine electrode position or perforation through the ventricular septum into an adjacent heart chamber. In such alternative embodiments, one or two of cardiac conduction system capture, injury of current, and impedance may be used to determine electrode position or perforation through the ventricular septum.

In some embodiments, one of cardiac conduction system capture, injury of current, and impedance may be determined prior to determining the other variables being used. For example, cardiac conduction system capture may be determined first, then injury of current, and then impedance. In further examples, any combination or order of determination may be made.

During implantation of the implantable electrode, or during implantation of an implantable lead, in some examples, if the injury of current decreases, if the cardiac conduction system capture threshold starts to increase, and if the impedance decreases, then it may be determined that the implantable electrode, or implantable lead, is implanted in the interventricular septal wall proximate the LBB and prior to perforation into the LV chamber. The increase in the cardiac conduction system capture threshold may be any measurable increase equal to or greater than 1.0 volts. The decrease in the injury of current from a relatively higher amplitude to a relatively lower amplitude may be any measurable decrease. The decrease in the impedance may be any measurable decrease equal to or greater than 100 ohms. A first notification (e.g., audio, visual, etc.) may be issued, or initiated, in response to determining that the implantable electrode, or implantable lead, is implanted in the interventricular septal wall proximate the cardiac conduction system and prior to perforation of the LV chamber.

During continued advancement of the implantable electrode, or implantable lead, within the ventricular septum, in some examples, if the injury of current decreases further or disappears, if the cardiac conduction system capture threshold increases further or if there is loss of capture of the cardiac conduction system, and if the impedance decreases further, then it may be determined that the implantable electrode, or implantable lead, has perforated into the LV chamber. As discussed herein, “further” is relative to the value determined at or near the cardiac conduction system, or the value determined at a location understood as at or near the cardiac conduction system. The further increase in the cardiac conduction system capture threshold may be any further measurable increase greater than or equal to 1.0 volts. The further decrease in the injury of current may be any further measurable decrease greater than or equal to 1 millivolt (mV). The further decrease in the impedance may be any further measurable decrease greater than or equal to 100 ohms. A second notification may be issued, or initiated, in response to determining that the implantable electrode, or implantable lead, perforated through the ventricular septum into the LV chamber.

In one or more embodiments, illustrative systems, devices, and methods are described herein to determine implantable electrode position within the ventricular septum in “real time,” so as to provide effective cardiac conduction system therapy to a patient and so as to avoid electrode perforation into the LV chamber, which may harm heart tissue and may lead to electrode repositioning.

One illustrative system may be for use in assisting implantation of an implantable electrode. The system may include an implantable electrode configured to deliver cardiac conduction system pacing proximate a portion of a patient's cardiac conduction system. The system may include an external electrode configured to at least sense electrical activity of the patient's heart. The system may include a computing apparatus comprising processing circuitry. The computing apparatus may be operably coupled to the implantable electrode and the external electrode. The computing apparatus may be configured to, during implantation of the implantable electrode: monitor internal electrical activity using the implantable electrode during implantation of the implantable electrode. The computing apparatus may be further configured to monitor external electrical activity using the external electrode during implantation of the implantable electrode. The computing apparatus may be further configured to determine cardiac conduction system capture based on at least one of the internal and external monitored electrical activity during implantation of the implantable electrode. The computing apparatus may be further configured to determine injury of current based on at least one of the monitored internal and external electrical activity during implantation of the implantable electrode. The computing apparatus may be further configured to determine impedance based on at least one of the monitored internal and external electrical activity during implantation of the implantable electrode. The computing apparatus may be further configured to issue a first notification in response to determining that the implantable electrode is implanted in the interventricular septal wall proximate the cardiac conduction system and prior to perforation into the left ventricular (LV) chamber based on the cardiac conduction system capture, the injury of current, and the impedance.

One illustrative method may be to assist in implanting an implantable electrode comprising an implantable electrode proximate a patient's cardiac conduction system. The method may include, during implantation of the implantable electrode: monitoring internal electrical activity using the implantable electrode during implantation of the implantable electrode. The method may further include monitoring external electrical activity using an external electrode during implantation of the implantable electrode. The method may further include determining cardiac conduction system capture based on at least one of the internal and external monitored electrical activity during implantation of the implantable electrode. The method may further include determining injury of current based on at least one of the monitored internal and external electrical activity during implantation of the implantable electrode. The method may further include determining impedance based on at least one of the monitored internal and external electrical activity during implantation of the implantable electrode. The method may further include issuing a first notification in response to determining that the implantable electrode is implanted in the interventricular septal wall proximate the cardiac conduction system and prior to perforation into the left ventricular (LV) chamber based on the cardiac conduction system capture, the injury of current, and the impedance.

Another illustrative system may be for use in assisting implantation of an implantable electrode. The system may include an implantable electrode configured to deliver cardiac conduction system pacing proximate a portion of a patient's cardiac conduction system. The system may include a computing apparatus comprising processing circuitry. The computing apparatus may be operably coupled to the implantable electrode. The computing apparatus may be configured to, during implantation of the implantable electrode: monitor internal electrical activity using the implantable electrode during implantation of the implantable electrode. The computing apparatus may be further configured to determine cardiac conduction system capture, injury of current, and impedance based on the monitored internal electrical activity during implantation of the implantable electrode. The computing apparatus may be further configured to issue a first notification in response to determining that the implantable electrode is implanted in the interventricular septal wall proximate the cardiac conduction system and prior to perforation into the left ventricular (LV) chamber based on at least one of the determined cardiac conduction system capture, the injury of current, and the impedance.

Another illustrative method may be to assist in implanting an implantable electrode comprising an implantable electrode proximate a patient's cardiac conduction system. The method may include, during implantation of the implantable electrode: monitoring internal electrical activity using the implantable electrode during implantation of the implantable electrode. The method may further include determining cardiac conduction system capture, injury of current, and impedance, based the monitored internal electrical activity during implantation of the implantable electrode. The method may further include issuing a first notification in response to determining that the implantable electrode is implanted in the interventricular septal wall proximate the cardiac conduction system and prior to perforation into the left ventricular (LV) chamber based on at least one of the cardiac conduction system capture, the injury of current, and the impedance.

The above summary is not intended to describe each embodiment or every implementation of the present disclosure. A more complete understanding will become apparent and appreciated by referring to the following detailed description and claims taken in conjunction with the accompanying drawings.

### BRIEF DESCRIPTION OF THE DRAWINGS

FIG. 1 is a schematic diagram of a heart and cardiac conduction system of a patient.

FIG. 2A is a conceptual diagram illustrating an illustrative therapy system that is configured to provide cardiac conduction system pacing therapy to the left bundle branch using a single lead placed in the right ventricle.

FIG. 2B is a close-up view of the lead in the patient's heart of FIG. 2A.

FIG. 3 is a functional diagram illustrating an example of a configuration of an implantable medical device, programmer, and external electrode apparatus of FIGS. 2A-2B.

FIG. 4 is a block diagram of an illustrative method of determining that an implantable electrode is proximate the cardiac conduction system, such as the lead of FIGS. 2A-B.

FIG. 5 is a block diagram of an illustrative method of determining a change in the injury of current during electrode implantation.

FIG. 6 is a block diagram of an illustrative method of determining a change in the cardiac conduction system capture threshold during electrode implantation.

FIG. 7 is a block diagram of an illustrative method of determining a change in the impedance during electrode implantation.

FIG. 8 is a block diagram of an illustrative method of implanting an implantable electrode such as the electrodes of FIGS. 2A-B based on real-time monitoring of perforation-related variables.

FIG. 9 is a block diagram of an illustrative method of implanting an implantable electrode such as the electrodes of FIGS. 2A-B.

FIG. 10 is a block diagram of an illustrative method of implanting an implantable electrode such as the electrodes of FIGS. 2A-B.

### DETAILED DESCRIPTION

In the following detailed description of illustrative embodiments, reference is made to the accompanying figures of the drawing which form a part hereof, and in which are shown, by way of illustration, specific embodiments which may be practiced. It is to be understood that other embodiments may be utilized, and structural changes may be made without departing from (e.g., still falling within) the scope of the disclosure presented hereby.

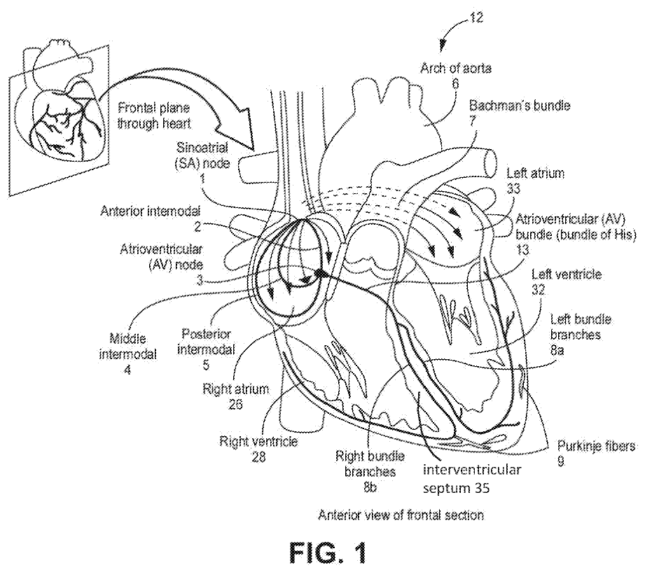

Illustrative systems, devices, and methods shall be described with reference to FIGS. 1-10. It will be apparent to one skilled in the art that elements or processes from one embodiment may be used in combination with elements or processes of the other embodiments, and that the possible embodiments of such systems, devices, and methods using combinations of features set forth herein is not limited to the specific embodiments shown in the Figures and/or described herein. Further, it will be recognized that the embodiments described herein may include many elements that are not necessarily shown to scale. Still further, it will be recognized that timing of the processes and the size and shape of various elements herein may be modified but still fall within the scope of the present disclosure, although certain timings, one or more shapes and/or sizes, or types of elements, may be advantageous over others.

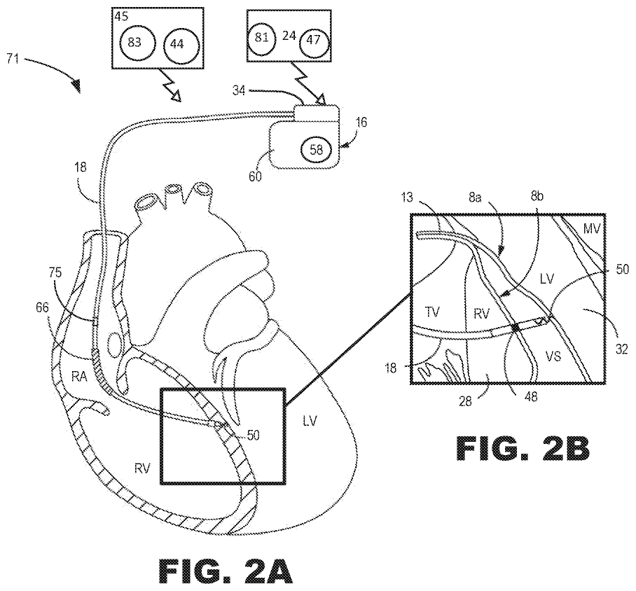

FIG. 1 depicts a schematic diagram of a heart 12 and cardiac conduction system, and FIGS. 2A-B depict a conceptual diagram showing illustrative therapy systems 71 that is configured to provide cardiac conduction system pacing therapy to the LBB using a cardiac conduction pacing therapy lead 18 that may be implanted in the heart 12 of a patient. In alternative embodiments, a leadless pacing device may be used as described herein. The patient ordinarily, but not necessarily, will be a human. The therapy system 71 may include IMD 16, which is coupled to the cardiac conduction pacing therapy lead 18 (e.g., left bundle branch pacing lead, right bundle branch pacing lead, His-bundle pacing lead, etc.) and a programmer 24. The IMD 16 may be, for example, an implantable pacemaker, cardioverter, and/or defibrillator that provides electrical pulses to the heart 12 via electrodes coupled to the cardiac conduction pacing therapy lead 18. Further non-limiting examples of the IMD 16 include the following: a pacemaker with a medical lead, an implantable cardioverter-defibrillator (ICD), an intracardiac device, a leadless pacing device (LPD), a subcutaneous ICD (S-ICD), and a subcutaneous medical device (e.g., nerve stimulator, inserted monitoring device, etc.).

The cardiac conduction pacing therapy lead 18 may extend into the heart 12 of the patient to sense electrical activity of the heart 12 and/or deliver electrical stimulation to the heart 12. In the example shown in FIG. 2A, the cardiac conduction system pacing therapy lead 18 extends through one or more veins and the vena cava, the right atrium 26, through the tricuspid valve and into the right ventricle 28 of the heart 12 to pace the cardiac conduction system (e.g., within the ventricular septal wall 35, proximate and/or in direct contact with the left bundle branch 8a, proximate and/or in direct contact with the right bundle branch 8b, proximate and/or in direct contact with the His bundle 13, etc.). In some embodiments, the cardiac conduction system pacing therapy lead 18 may be positioned within about 1 millimeter of a portion of the cardiac conduction system such as, e.g., the left bundle branch 8a. The cardiac conduction system pacing therapy lead 18 may be positioned for positioning electrodes 48, 50 near, adjacent, on, within, or around the RBB, LBB (respectively) for sensing electrocardiogram signals and pacing the cardiac conduction system. The cardiac conduction system pacing therapy lead 18 is shown with a ring electrode 48 and a helix tip electrode 50 that may be selected in various bipolar pacing electrode pairs for pacing the RBB and the LBB (respectively) and for sensing RBB and LBB electrocardiogram signals (respectively). One of the electrodes 48, 50 may be selected in combination with IMD housing 60 or a coil electrode 66 for delivering unipolar RBB and LBB pacing and/or sensing unipolar RBB and LBB electrocardiogram signals. In alternative embodiments, the cardiac conduction system pacing therapy lead 18 is also used to pace the RA using an electrode 75 (shown in FIG. 2A), or is used to pace the RA in addition to the cardiac conduction system.

One example of a cardiac conduction system pacing therapy lead (e.g., a His lead) can be the SELECTSECURE™ 3830. A description of the SELECTSECURE™ 3830 is found in the Medtronic model SELECTSECURE™ 3830 manual (2013), incorporated herein by reference in its entirety. The SELECTSECURE™ 3830 includes two conductors without lumens.

As used herein, cardiac conduction system pacing therapy refers to any techniques that are configured to deliver pacing therapy (e.g., pacing pulses, electrical stimulation, etc.) to the cardiac conduction system including, e.g., the His bundle 13, the left bundle branch 8a, the right bundle branch 8b, etc., in order to initiate activation. As used herein, the term “activation” refers to a sensed or paced event. For example, an atrial activation may refer to an atrial sense or event (As) or an atrial pace or artifact of atrial pacing (Ap). As will be described herein, an atrial sense may be detected, or identified, in one or more various signals monitored using one or more various devices or sensors located in one or more various locations. For example, an atrial sense may be detected in a near-field electrical signal using an electrode positioned in the right atrium with a respective reference electrode (e.g., an electrode on the housing of the implantable medical device). Further, for example, an atrial sense may be detected in a far-field electrical signal using electrodes positioned outside of the right atrium such as in the right ventricle or ventricular septum and a respective reference electrode. Still, for example, an atrial sense may be detected in a far-field signal using a mechanical cardiac activation sensor such as an accelerometer or microphone (e.g., a heart sound sensor) positioned outside of the right atrium such as in the right ventricle or ventricular septum or another portion of the patient's body (e.g., within the can or housing of an IMD positioned outside of the patient's heart). Similarly, a ventricular activation may refer to a ventricular sense or event (Vs) or a ventricular pace or artifact of ventricular pacing (Vp), which may be described as ventricular stimulation pulses. In some embodiments, an activation interval can be detected from As or Ap to Vs or Vp, as well as Vp to Vs. In particular, activation intervals may include a pacing (Ap or Vp) to ventricular interval (left ventricular or right ventricular sense) or an atrial-sensing (As) to ventricular-sensing interval (left ventricular or right ventricular).

Illustrative IMDs may be described as delivering one or both of conventional pacing therapy and cardiac conduction system pacing therapy. Conventional, or traditional, pacing therapy may be described as delivering pacing pulses into myocardial tissue that is not part of the cardiac conduction system of the patient's heart such that, e.g., the pacing pulses trigger electrical activation that propagates primarily from one myocardial cell to another myocardial cell (also referred to as “cell-to-cell”) as opposed to propagating within the cardiac conduction system prior to the myocardial tissue. For instance, conventional pacing therapy may deliver pacing pulses directly into the muscular heart tissue (e.g., myocardial tissue) that is to be depolarized to provide the contraction of the heart. For example, conventional left ventricular pacing therapy may utilize a left ventricular coronary sinus lead that is implanted so as to extend through one or more veins, the vena cava, the right atrium 26, and into the coronary sinus to a region adjacent to the free wall of the left ventricle 32 of the heart 12 so as to deliver pacing pulses to the myocardial tissue of the free wall of the left ventricle 32.

Illustrative cardiac conduction system pacing therapy may be described in, for example, U.S. Pat. App. Pub. No. 2019/0111270 A1 entitled “His Bundle and Bundle Branch Pacing Adjustment” published on Apr. 18, 2019, which is incorporated herein by reference in its entirety. Illustrative left ventricular septal pacing may be described in, for example, U.S. patent application Ser. No. 16/521,000 entitled “AV Synchronous Septal Pacing” filed on Jul. 24, 2019, which is incorporated herein by reference in its entirety.

One or more elongated conductors of cardiac conduction pacing therapy lead 18 may extend through a hermetic feedthrough assembly, and within an insulative tubular member of the respective lead, and may electrically couple an electrical pulse generator (contained within housing) to one or more electrodes such as, e.g., ring electrodes, tips electrodes, helical electrodes, etc. The conductors may be formed by one or more electrically conductive wires comprising, for example, MP35N alloy known to those skilled in the art, in a coiled or cabled configuration, and the insulative tubular member may be any suitable medical grade polymer, for example, polyurethane, silicone rubber, or a blend thereof. According to one or more illustrative embodiments, the flexible lead body may extend a pre-specified length (e.g., about 10 centimeters (cm) to about 20 cm, or about 15 to 20 cm) from a proximal end to a distal end. The lead body may be less than about 7 French (FR) but typically in the range of about 3 FR to 4 FR in size. In one or more embodiments, about 2 FR size to about 3 FR size lead body is employed.

Cardiac conduction system pacing may include at least one of His bundle pacing, LBB pacing, and RBB pacing. Bundle branch pacing may bypass the pathological region and may have a low and stable pacing threshold. In some embodiments, only one of the left bundle branch or the right bundle branch may be paced using one or more pacing leads. In further embodiments, both bundle branches may be paced at the same time (e.g., dual bundle branch pacing), which may mimic intrinsic activation propagation via the His bundle-Purkinje conduction system, e.g., paced activation propagates via both bundle branches to both ventricles for synchronized contraction. His bundle pacing, on the other hand, typically paces the His bundle proximal to the bundle branches. In some embodiments, the IMD 16 may be coupled to one, two, or more electrodes located in one or more bundle branches configured for bundle branch pacing.

In some embodiments, the IMD 16 may be an intracardiac pacemaker or leadless pacing device (LPD) configured to pace one or more portions of the cardiac conduction system such as one or both of the bundle branches. As used herein, “leadless” refers to a device being free of a lead extending out of the heart 12. In other words, a leadless device may have a lead that does not extend from outside of the heart to inside of the heart. Some leadless devices may be introduced through a vein, but once implanted, the leadless devices are free of, or may not include, any transvenous lead and may be configured to provide cardiac therapy without using any transvenous lead. In one or more embodiments, an illustrative LPD for bundle pacing does not use a lead to operably connect to an electrode disposed proximate to the septum when a housing of the device is positioned in the atrium. A leadless electrode may be leadlessly coupled to the housing of the medical device without using a lead between the electrode and the housing. For example, the cardiac conduction pacing therapy lead 18 of FIG. 2 may not be required, and instead electrodes 48, 50, 66, and 75 may be implanted in the illustrated locations without the use of the cardiac conduction pacing therapy lead 18.

The IMD 16 may sense electrical signals attendant to the depolarization and repolarization of the heart 12 via various electrodes as shown in FIG. 2A coupled to cardiac conduction pacing therapy lead 18. In some examples, the IMD 16 provides pacing pulses to the heart 12 based on the electrical signals sensed within the heart 12. The configurations of the electrodes used by the IMD 16 for sensing and pacing may be unipolar or bipolar.

The IMD 16 may also provide defibrillation therapy and/or cardioversion therapy via electrodes located on cardiac conduction pacing therapy lead 18. For example, the IMD 16 may detect atrial arrhythmias of heart 12, such as atrial fibrillation of the atria 26, 33, and then may deliver defibrillation therapy to the heart 12 in the form of electrical pulses. Also, the IMD 16 may detect ventricular arrhythmias of the heart 12, such as ventricular fibrillation of the ventricles 28, 32, and then may deliver defibrillation therapy to the heart 12 in the form of electrical pulses. In some examples, the IMD 16 may be programmed to deliver a progression of therapies, e.g., pulses with increasing energy levels, until fibrillation of the heart 12 is stopped. The IMD 16 may detect fibrillation employing one or more fibrillation detection techniques known in the art.

In some examples, the programmer 24 as shown in FIGS. 2A-B may be a handheld computing device or a computer workstation or a mobile phone. The programmer 24 may include a user interface that receives input from a user. The user interface may include, for example, a keypad and a display 47, which may for example, be a cathode ray tube (CRT) display, a liquid crystal display (LCD) or light emitting diode (LED) display. The keypad may take the form of an alphanumeric keypad, or a reduced set of keys associated with particular functions. The programmer 24 can additionally or alternatively include a peripheral pointing device, such as a mouse, via which a user may interact with the user interface. In some embodiments, the display 47 of the programmer 24 may include a touch screen display, and a user may interact with the programmer 24 via the display 47. The display 47 may be operatively couplable to a processor as described herein. Through the graphical user interface on the programmer 24, a user may configure one or more pacing therapies, select one or more pacing modes, etc.

In some embodiments, the programmer 24 may include similar processing components 81 as described with respect to the IMD 16 as discussed further herein (e.g., sensing module, stimulation generator, processor, telemetry module, memory, and power source, together noted as reference number 81). The processing components 81 can receive the EGM signal(s) from one or more of the electrode(s) 48, 50, 58, 66, and 75. The processing components 81 can receive the ECG signal(s) from one or more of the external electrode(s) 44. In some embodiments, the programmer 24 may be in wired or wireless communication with the IMD 16 and/or external electrode apparatus 45, as described further herein.

Additionally, various pacing settings may be adjusted, or configured, based on various sensed signals. For example, various near-field and far-field signals may be sensed by one or more of the electrodes coupled to the IMD 16 and/or other devices operatively coupled thereto. For example, right ventricular depolarization and left ventricular depolarization intervals may be monitored or measured within a near-field or far-field signal and then may be used to adjust, configure, and select cardiac conduction system pacing therapy. Further, for example, QRS morphology (e.g., QRS peak, various QRS intervals, ST interval, amplitude, etc.) may be monitored or measured within a near-field or far-field signal and then may be used to adjust, configure, and select cardiac conduction system pacing therapy. Still further, for example, one or more of right ventricular depolarization and left ventricular depolarization interval consistency, and QRS morphology consistency may be monitored or measured within a near-field or far-field signal and then may be used to adjust, configure, and select cardiac conduction system pacing therapy.

The illustrative therapy systems described herein such as IMD 16 may be utilized to deliver cardiac conduction system pacing therapy according to a variety of different modes such as, e.g., inhibited pacing mode, ventricular fusion pacing mode, atrioventricular synchronous pacing mode, atrial fibrillation pacing mode, etc.

As used herein, the term “far-field” electrical signal refers to the result of measuring cardiac activity using a sensor, such as an electrode, positioned outside of an area of interest. For example, a far-field electrical signal representing electrical activity of a chamber of interest of the patient's heart may be measured from an electrode positioned in an adjacent chamber (i.e., a chamber different from than that of the chamber of interest that is next to or near the chamber of interest). More specifically, for example, atrial electrical activity, or electrical activity originating one or more both atria, representative of depolarization of the one or both atria may be monitored in a far-field electrical signal measured using an electrode positioned outside of the right atrium such as in the right or left ventricle, or in the ventricular septum. As used herein, the term “near-field” electrical signal refers to the result of measuring cardiac activity using a sensor, such as an electrode, positioned near an area of interest. For example, an electrical signal measured using an electrode positioned on the left side of the patient's ventricular septum is one example of a near-field electrical signal of the patient's LV.

A user, such as a physician, technician, or other clinician, may interact with the programmer 24 to communicate with the IMD 16. For example, the user may interact with the programmer 24 to retrieve physiological or diagnostic information from the IMD 16. Additionally, a user may also interact with the programmer 24 to program the IMD 16, e.g., select values for operational parameters of the IMD 16. The IMD 16 and programmer 24 may communicate via wireless communication using any techniques known in the art. Examples of communication techniques may include, for example, low frequency or radiofrequency (RF) telemetry, but other techniques are also contemplated. In some examples, the programmer 24 may include a programming head that may be placed proximate to the patient's body near the IMD 16 implant site in order to improve the quality or security of communication between the IMD 16 and the programmer 24.

The cardiac conduction pacing therapy lead 18 may be electrically coupled to a stimulation generator, a sensing module, or other modules of IMD 16 via connector block 34. In some examples, proximal ends of cardiac conduction pacing therapy lead 18 may include electrical contacts that electrically couple to respective electrical contacts within the connector block 34. In addition, in some examples, the cardiac conduction pacing therapy lead 18 may be mechanically coupled to the connector block 34 with the aid of set screws, connection pins, or another suitable mechanical coupling mechanism.

While the cardiac conduction system pacing therapy lead 18 is shown and described with respect to FIGS. 2A-B as being placed in the RV along the intraventricular septal wall 35, in other examples, the cardiac conduction system pacing therapy lead 18 may be placed in the right atrium within the triangle of Koch region (not shown) with the corresponding electrodes 48, 50 tunneled through the septal tissue to be positioned proximate the RBB and LBB, respectively. In such examples, the system may not contain a lead positioned within the RV, yet still obtain the benefit of LBB or RBB pacing and sensing as described herein. Additionally or alternatively, the system in such examples may include an additional lead or electrode(s) positioned in the RA configured to pace the RA that may be different from the cardiac conduction system pacing therapy lead 18 or the respective LBB and RBB electrodes.

Cardiac conduction pacing therapy lead 18 includes an elongated, insulative lead body, which may carry any number of conductors. In the illustrated example, bipolar electrodes 48 and 50 are located proximate to a distal end of the cardiac conduction system pacing therapy lead 18. An optional pressure sensor (not shown) may respond to an absolute pressure inside RV, or may be positioned within other regions of the heart 12 or elsewhere within or proximate to the cardiovascular system of the patient to monitor cardiovascular pressure associated with mechanical contraction of the heart. In addition, in some examples, the optional pressure sensor may be self-contained device that is implanted within the heart 12 and wirelessly correspond with the IMD 16.

The electrode 48 may take the form of a ring electrode, and the electrode 50 may take the form of extendable and/or fixed helix tip electrodes mounted within the insulative electrode heads. Each of the electrodes 48 and 50 may be electrically coupled to a respective one of the coiled conductors within the lead body and thereby coupled to the respective one of the electrical contacts on the proximal end of cardiac conduction pacing therapy lead 18.

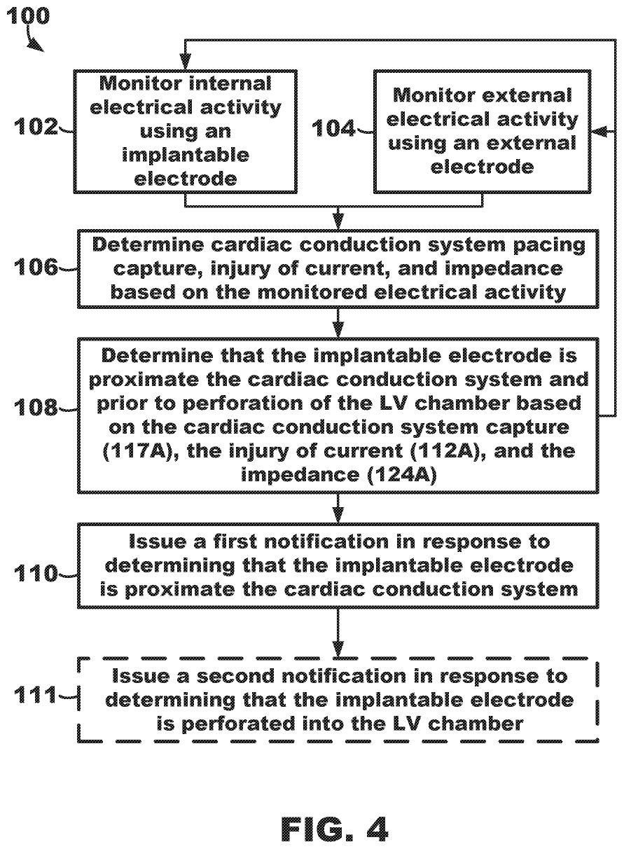

The electrodes 48 and 50 may sense electrical signals attendant to the depolarization and repolarization of the heart 12. The electrical signals are conducted to the IMD 16 via cardiac conduction pacing therapy lead 18. In some examples, the IMD 16 also delivers pacing pulses via the electrodes 48, 50 to cause depolarization of cardiac tissue of heart 12, in particular, by delivering pacing pulses to the cardiac conduction system. In some examples, as illustrated in FIG. 2A, the IMD 16 may include one or more housing electrodes, such as housing electrode 58, which may be formed integrally with an outer surface of a hermetically sealed housing 60 of the IMD 16 or otherwise coupled to the housing 60. In some examples, the housing electrode 58 may be defined by an uninsulated portion of an outward facing portion of the housing 60 of the IMD 16. Other divisions between insulated and uninsulated portions of housing 60 may be employed to define two or more housing electrodes. In some examples, the housing electrode 58 includes substantially all of the housing 60. Any of the electrodes 48, 50 may be used for unipolar sensing or pacing in combination with the housing electrode 58 or for bipolar sensing with two electrodes in the same pacing lead. In one or more embodiments, the housing 60 may enclose a stimulation generator (see FIG. 4) that generates cardiac pacing pulses and defibrillation or cardioversion shocks, as well as a sensing module for monitoring the patient's heart rhythm.

The cardiac conduction pacing therapy lead 18 may also include elongated electrode 66 (shown in FIG. 2A), which may take the form of a coil. The IMD 16 may deliver defibrillation shocks to the heart 12 via the elongated electrode 66 and the housing electrode 58. The electrodes 58, 66 may also be used to deliver cardioversion pulses to the heart 12. The electrode 66 may be fabricated from any suitable electrically conductive material, such as, but not limited to, platinum, platinum alloy or other materials known to be usable in implantable defibrillation electrodes. In alternative embodiments, the electrodes 48, 50, 58, 66 may be implanted using a leadless pacing device as opposed to the cardiac conduction pacing therapy lead 18.

The elongated electrodes may be selected in a unipolar electrode vector with any of the lead-based tip or ring electrodes for sensing unipolar electrocardiogram signals for analysis and determination of ventricular conduction conditions. In some instances, the elongated electrodes may be used with the housing 60 for sensing a far-field electrocardiogram signal for use in determining atrial depolarizations or activations, etc.

In other embodiments (not shown), additional examples of dual chamber and triple chamber therapy systems may be utilized. Such examples may use two or three or more leads, or various leadless devices and electrodes. In a dual chamber example, electrodes may be implanted within the RV and the RA to pace one or more portions of the cardiac conduction system such as the His bundle or one or both bundle branches, and to pace the RA, respectively.

Electrode 50 may take the form of a helix (also referred to as a helical electrode) that may be positioned proximate to, near, adjacent to, or in, area or portions of the cardiac conduction system such as, e.g., ventricular septum, triangle of Koch, the His bundle, left bundle branch tissues, and/or right bundle branch tissue. The cardiac conduction system pacing lead 18 may be configured as a bipolar lead that may be used with a pacemaker device, a CRT-P device, or a CRT-ICD. As shown, the cardiac conduction system pacing lead 18 may be advanced into the RV chamber of the heart into the ventricular septum to achieve the ideal positioning of the electrode 50. During electrode advancement, the electrode 50 may be advanced too far into the ventricular septum such that the electrode 50 perforates through the ventricular septum and into another chamber of the heart (e.g., the LV).

In particular, FIGS. 2A-2B show the patient's heart 12 implanted with cardiac conduction system pacing lead 18 to deliver bundle branch pacing according to one example of the single chamber therapy system 71. The cardiac conduction system therapy lead 18 is positioned, or located, through the tricuspid valve into the RV and implanted in the interventricular septum, e.g., about 1 to 2 centimeters in an apical direction away from the RA (as illustrated in FIGS. 2A-B). FIG. 2B is a close-up view of the cardiac conduction system therapy lead 18 in the patient's heart 12 of FIG. 2A. In some embodiments, the cardiac conduction system therapy lead 18 may be the only lead implanted in the heart 12. In other embodiments as discussed herein, there may be leads in addition to the cardiac conduction system therapy lead 18 implanted in the heart 12. The one or more implantable electrodes of the cardiac conduction system therapy lead 18 may include a pacing electrode implantable proximate the cardiac conduction system to deliver cardiac conduction system pacing therapy. In alternative embodiments, leadless pacing devices and electrodes may be used as described herein.

As illustrated, the cardiac conduction system pacing therapy lead 18 is implanted in the interventricular septal wall 35, or ventricular septum, from the RV toward the LV. The cardiac conduction system pacing therapy lead 18 may not pierce through the wall of the LV or extend into the LV chamber. The electrodes 48 and 50 may be disposed on a distal end portion of the cardiac conduction system pacing therapy lead 18 as discussed herein at least with respect to FIG. 2A. However, during electrode advancement, the electrodes 48, 50 may also be advanced such that one or both of the electrodes 48, 50 undesirably perforates through the ventricular septum and into another chamber of the heart (e.g., the LV). This disclosure generally relates to determining whether an implantable electrode is positioned within the ventricular septum of the heart of a patient near or at the cardiac conduction system, and whether the implantable electrode should be repositioned based on that determination. The present disclosure further relates to determining that the implantable electrode has perforated the ventricular septum into the LV chamber, and that the implantable electrode should be repositioned based on that determination.

Prior to reaching the ventricular septum, the implantable electrode(s) 48, 50 are advanced into the patient and electrical activity may be monitored using the external electrode apparatus 45 and resultant ECG signal. Once the ventricular septum is reached, the implantable electrode(s) 48, 50 may be connected to the programmer 24 as described herein. Thereafter, the monitored electrical activity may include internal and external monitored electrical activity as discussed herein.

The implantable electrode(s) 48, 50 advance through the septum as the user advances the implantable electrode farther into the patient. In embodiments with a lead, such as the cardiac conduction system pacing therapy lead 18, the electrode(s) 48, 50 are advanced into the patient as the lead is advanced into the patient. The cardiac conduction system pacing therapy lead 18, or the implantable electrode(s) 48, 50, may be advanced into the ventricular septum via rotation, and in embodiments where electrode 50 is a helix, such rotation will rotate the electrode 50 and advance it further into the ventricular septal tissue. Rotation of the implantable electrode(s) 48, 50, or rotation of the cardiac conduction system pacing therapy lead 18, may be effected by using a rotatable coupler connected to the implantable electrode(s) or to the cardiac conduction system pacing therapy lead 18. The rotatable coupler may allow for continuous monitoring of electrical signals using the programmer 24 and computing apparatus 81, via the electrode(s) 48, 50 while they are being rotated and advanced. Illustrative rotatable couplers may be described in, for example, U.S. Pat. App. Pub. No. 2022/0088395 A1, entitled “Rotatable Adapter For Connecting Implantable Medical Leads To Test Devices” published on Mar. 24, 2022, which is incorporated herein by reference in its entirety.

The cardiac conduction system pacing therapy lead 18 may also be described as a shaft. The electrodes 48 and 50 may be the same as or similar to electrode 48 and electrode 50 shown in FIG. 2A and the electrode 48 is configured to sense or pace the right bundle branch and the electrode 50 is configured to sense or pace the left bundle branch, for example, during dual bundle branch pacing. Accordingly, the electrode 48 may be implanted near right bundle branch 8b, and the electrode 50 may be implanted near the left bundle branch 8a. The electrode 50 may be implanted towards the left side of the patient's ventricular septum. The electrode 48 may be implanted towards the right side of the patient's ventricular septum. In one embodiment, the electrode 50 may be a helix electrode, and the electrode 48 may be a ring electrode. As described herein, in alternative embodiments a leadless pacing device may be used. In such alternative embodiments, the electrodes 48, 50 may be implanted in the locations shown and described, without the cardiac conduction system pacing therapy lead 18.

During dual bundle branch pacing, both the electrodes 48 and 50 may each deliver a pulse to achieve synchronized activation, or excitation, of the right bundle branch 8b and the left bundle branch 8a, which may result in synchronized activation of the RV and the LV. In some embodiments, the pulses may be delivered at the same time to achieve synchrony. In other embodiments, the pulses may be delivered with a delay to achieve synchrony.

Although the cardiac conduction system pacing therapy lead 18 as shown in configured for dual bundle branch pacing using the electrodes 48, 50, it is to be understood that the cardiac conduction system pacing therapy lead 18 or leads similar thereto are considered herein that may only include one of the electrode 48 and the electrode 50, and thus, only configured to deliver cardiac conduction system pacing therapy to one of the right bundle branch and the left bundle branch. In alternative embodiments, both electrodes 48 and 50 may be located on the cardiac conduction system pacing therapy lead 18, but the IMD 16 may use just one of electrodes 48, 50 to pace only one bundle branch.

Additionally, the cardiac conduction system pacing therapy lead 18 may include an RA electrode 75 disposed more proximal to the electrodes 48, 50 along the cardiac conduction system pacing therapy lead 18. The RA electrode 75 may be positioned in or near the RA and may function as an anode for cathodal pulses from the electrode 48 and/or the electrode 50. Further, the RA electrode 75 may provide atrial sensing to, e.g., sense atrial depolarizations or activations, to sense or detect atrial fibrillation, etc. Although the cardiac conduction system pacing therapy lead 18 as shown includes the RA electrode 75, it is to be understood that the cardiac conduction system pacing therapy lead 18 may not include the RA electrode 75, and instead, only include one or both of the electrode 48 and the electrode 50. In alternative embodiments, the electrode 75 may be implanted using a leadless pacing device as opposed to the cardiac conduction pacing therapy lead 18.

As illustrated in FIG. 2, the therapy system 71 may further include an external electrode apparatus 45, which may include one or more external electrode(s) 44. The external electrode 44 may be configured to at least sense electrical activity of the patient's heart 12. The external electrode 44 may be further configured to deliver pacing (e.g., cardiac conduction system pacing). The external electrode apparatus 45 may include a body surface ECG apparatus (e.g., an ECG belt, an ECG vest, etc.), which may include a standard 12-lead ECG, a 2-lead ECG, a 1-lead ECG, or any other number of leads. The ECG apparatus may include use of one or more surface electrodes (e.g., external electrode(s) 44), including electrodes positioned on the surface of a patient near or at the standard I, II, III, IV, V, and VI chest leads and/or upper right and left arm and lower right and left leg limb leads, for example.

In some embodiments, the external electrode apparatus 45 may include similar processing components 83 as described with respect to the IMD 16 as discussed further herein (e.g., sensing module, stimulation generator, processor, telemetry module, memory, and power source, together noted as reference number 83). The processing components 83 can receive the EGM signal(s) from one or more of the electrode(s) 48, 50, 58, 66, and 75. The processing components 83 can receive the ECG signal(s) from one or more of the external electrode(s) 44. In some embodiments, the external electrode apparatus 45 may be in wired or wireless communication with the IMD 16 and/or programmer 24.

When the cardiac conduction system pacing therapy lead 18 is positioned for delivering bundle branch pacing, of one or both bundle branches, cardiac conduction system pacing therapy may be combined with traditional ventricular myocardial pacing of the left ventricle using, for example, a coronary sinus lead to correct a left ventricular conduction delay and achieve electrical and mechanical synchrony of the left and right ventricles. As such, in some examples, one or more processors, one or more processing circuits, or a computing apparatus of the IMD 16 may select a cardiac conduction system pacing therapy plus traditional left ventricular myocardial pacing therapy that includes, for example, single or bilateral bundle branch pacing, e.g., using the cardiac conduction system pacing therapy lead 18, combined with left ventricular myocardial pacing using the coronary sinus lead (not shown).

The configuration of therapy system 71 illustrated in FIG. 2 is merely an example. In other examples, a therapy system may include epicardial leads and/or patch electrodes instead of or in addition to the cardiac conduction system pacing therapy lead 18 or other configurations shown or described herein or incorporated by reference. Further, the IMD 16 need not be implanted within patient. As such, it is to be understood that the illustrative therapy systems described herein may include any suitable number of leads coupled to IMD 16, and each of the leads may extend to any location within or proximate to the heart 12. For example, illustrative therapy systems may include a single transvenous lead located as illustrated in FIGS. 2A-2B, or two or more transvenous leads located in various chambers.

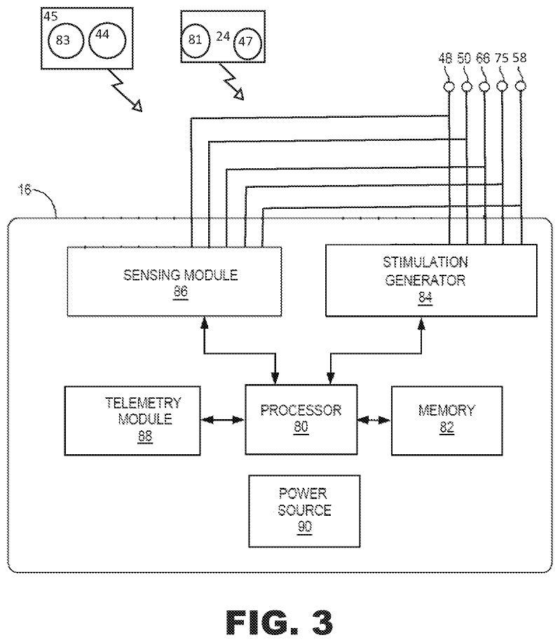

FIG. 3 is a functional diagram of one example configuration of the IMD 16, the external electrode apparatus 45, the programmer 24, and electrodes 44, 48, 50, 58, 66, and 75. The IMD 16 includes a computing apparatus 80 (which may include a processor), a memory 82, a stimulation generator 84 (e.g., electrical pulse generator or signal generating circuit), a sensing module 86 (e.g., sensing circuit), a telemetry module 88, and a power source 90. One or more components of the IMD 16, such as the computing apparatus 80, may be contained within a housing of the IMD 16 (e.g., within a housing of a pacemaker). The telemetry module 88, the sensing module 86, or both the telemetry module 88 and the sensing module 86 may be included in a communication interface. The memory 82 includes computer-readable instructions that, when executed by the processor of the computing apparatus 80, cause the IMD 16 and the computing apparatus 80 to perform various functions attributed to the IMD 16 and the computing apparatus 80 herein. The memory 82 may include any volatile, non-volatile, magnetic, optical, or electrical media, such as a random-access memory (RAM), read-only memory (ROM), non-volatile RAM (NVRAM), electrically erasable programmable ROM (EEPROM), flash memory, or any other digital media. In some embodiments, the programmer 24 and/or external electrode apparatus 45 include a similar memory to the memory 82 as described herein.

The computing apparatus 80 may include any one or more of a microprocessor, a controller, a digital signal processor (DSP), an application specific integrated circuit (ASIC), a field-programmable gate array (FPGA), or equivalent discrete or integrated logic circuitry. In some examples, computing apparatus 80 may include multiple components, such as any combination of one or more microprocessors, one or more controllers, one or more DSPs, one or more ASICs, or one or more FPGAs, as well as other discrete or integrated logic circuitry. The functions attributed to computing apparatus 80 herein may be embodied as software, firmware, hardware, or any combination thereof. The computing apparatus 80 controls the stimulation generator 84 to select a therapy mode and deliver stimulation therapy to the heart 12 according to the selected pacing mode, which may be stored in the memory 82, and various sensing (e.g., atrial depolarizations or activations, ventricular atrial depolarizations or activations, heartrate, P-wave-to-R-wave intervals, etc.). Specifically, the computing apparatus 80 may control the stimulation generator 84 to deliver electrical pulses with amplitudes, pulse widths, frequency, or electrode polarities specified by the selected one or more therapy programs and therapy modes. The computing apparatus 80 may also monitor EGM signal(s) from the implantable electrode(s) 48, 50, 66, 75, and may also monitor ECG signal(s) from the external electrode(s) 44. The monitored ECG and/or EGM signals may be used to determine whether the implantable electrode is positioned within the ventricular septum near or at the cardiac conduction system (e.g., the LBB), and whether the implantable electrode should be repositioned based on that determination. The monitored ECG and/or EGM signals may be used to determine that the implantable electrode has perforated the ventricular septum into the LV chamber, and that the implantable electrode should be repositioned based on that determination. In some embodiments, the programmer 24 and/or external electrode apparatus 45 include a similar computing apparatus (81 and 83, respectively) to the computing apparatus 80 as described herein.

In some embodiments, the cardiac conduction system pacing lead 18 may be operably coupled to the electrode 75, which may be used to monitor or pace the RA. In some embodiments, the stimulation generator 84 may be electrically coupled to the electrodes 48, 50, 66, and 75, e.g., via conductors of the cardiac conduction pacing therapy lead 18 (or, in alternative embodiments, a leadless pacing device) or, in the case of housing electrode 58, via an electrical conductor disposed within the housing 60 of the IMD 16, or, in the case of external electrode(s) 44, via an electrical conductor disposed within the external electrode apparatus 45. The stimulation generator 84 may be configured to generate and deliver electrical stimulation therapy to the heart 12. For example, the stimulation generator 84 may deliver defibrillation shocks to the heart 12 via electrode 66. The stimulation generator 84 may deliver pacing pulses via the ring electrode 48 coupled to the cardiac conduction pacing therapy lead 18, and/or the helical electrodes 50 of the cardiac conduction pacing therapy lead 18. In various embodiments, the cardiac conduction system pacing therapy can be delivered through the cardiac conduction system pacing lead 18 that is connected to an atrial, right ventricular, or left ventricular connection port of the connector block 34. In some examples, the stimulation generator 84 delivers pacing, cardioversion, or defibrillation stimulation in the form of electrical pulses. In other examples, the stimulation generator 84 may deliver one or more of these types of stimulation in the form of other signals, such as sine waves, square waves, or other substantially continuous time signals. In some embodiments, the programmer 24 and/or external electrode apparatus 45 include a similar stimulation generator to the stimulation generator 84 as described herein.

The stimulation generator 84 may include a switch module and the computing apparatus 80 may use the switch module to select, e.g., via a data/address bus, which of the available electrodes are used to deliver defibrillation shocks or pacing pulses. The switch module may include a switch array, switch matrix, multiplexer, or any other type of switching device suitable to selectively couple stimulation energy to selected electrodes.

The sensing module 86 monitors signals from at least one of the electrodes 44, 48, 50, 58, 66, or 75 in order to monitor electrical activity of the heart 12, e.g., via electrical signals, such as electrocardiogram (ECG) signals and/or electrograms (EGMs). The sensing module 86 may also include a switch module to select which of the available electrodes are used to sense the heart activity. In some examples, the computing apparatus 80 may select the electrodes that function as sense electrodes via the switch module within the sensing module 86, e.g., by providing signals via a data/address bus. In some examples, the sensing module 86 includes one or more sensing channels, each of which may include an amplifier. In response to the signals from the computing apparatus 80, the switch module may couple the outputs from the selected electrodes to one of the sensing channels. In some embodiments, the programmer 24 and/or external electrode apparatus 45 include a similar sensing module to the sensing module 86 as described herein.

In some examples, one channel of the sensing module 86 may include an R-wave amplifier that receives signals from the electrodes 48, 50, which are used for pacing and sensing in the RV of the heart 12. In some examples, the R-wave amplifiers may take the form of an automatic gain-controlled amplifier that provides an adjustable sensing threshold as a function of the measured R-wave amplitude of the heart rhythm.

In addition, in some examples, one channel of the sensing module 86 may include a P-wave amplifier that receives signals from electrodes which are used for pacing and sensing in the RA of heart 12. In some examples, the P-wave amplifier may take the form of an automatic gain-controlled amplifier that provides an adjustable sensing threshold as a function of the measured P-wave amplitude of the heart rhythm. Examples of R-wave and P-wave amplifiers are described in U.S. Pat. No. 5,117,824 to Keimel et al., which issued on Jun. 2, 1992, and is entitled, “APPARATUS FOR MONITORING ELECTRICAL PHYSIOLOGIC SIGNALS,” and is incorporated herein by reference in its entirety. Other amplifiers may also be used. Furthermore, in some examples, one or more of the sensing channels of the sensing module 86 may be selectively coupled to the housing electrode 58, or the elongated electrode 66, or the RA electrode 75, with or instead of one or more of the electrodes 48 or 50, e.g., for unipolar sensing of R-waves or P-waves in any of the chambers 26, 28, or 32 of the heart 12.

In some examples, the sensing module 86 includes a channel that includes an amplifier with a relatively wider pass band than the R-wave or P-wave amplifiers or a high-resolution amplifier with relatively narrow-pass band for His bundle or bundle branch potential recording. Signals from the selected sensing electrodes that are selected for coupling to this wide-band amplifier may be provided to a multiplexer, and thereafter converted to multi-bit digital signals by an analog-to-digital converter for storage in the memory 82 as an electrogram (EGM). In some examples, the storage of such EGMs in the memory 82 may be under the control of a direct memory access circuit. The computing apparatus 80, 81, 83 may employ digital signal analysis techniques to characterize the digitized signals stored in memory 82 to detect and classify the patient's heart rhythm from the electrical signals. The computing apparatus 80, 81, 83 may detect and classify the heart rhythm of the patient by employing any of the numerous signal processing methodologies known in the art. The computing apparatus 81 may determine whether the implantable electrode (e.g., 48, 50) is positioned within the ventricular septum near or at the cardiac conduction system (e.g., the LBB) based on, e.g., cardiac conduction system capture, injury of current, impedance, etc., and whether the implantable electrode should be repositioned based on that determination. The computing apparatus 81 may determine that the implantable electrode has perforated the ventricular septum into the LV chamber, and that the implantable electrode should be repositioned based on that determination.

If the IMD 16 is configured to generate and deliver pacing pulses to the heart 12, the computing apparatus 80 may include pacer timing and control module, which may be embodied as hardware, firmware, software, or any combination thereof. The pacer timing and control module may include a dedicated hardware circuit, such as an ASIC, separate from other the computing apparatus 80 components, such as a microprocessor, or a software module executed by a component of the computing apparatus 80, which may be a microprocessor or ASIC. The pacer timing and control module may include programmable counters which control the basic time intervals associated with DDD, VVI, DVI, VDD, AAI, DDI, DDDR, VVIR, DVIR, VDDR, AAIR, DDIR and other modes of single and dual chamber pacing. In the aforementioned pacing modes, “D” may indicate dual chamber, “V” may indicate a ventricle, “I” may indicate inhibited pacing (e.g., no pacing), and “A” may indicate an atrium. The first letter in the pacing mode may indicate the chamber that is paced, the second letter may indicate the chamber in which an electrical signal is sensed, and the third letter may indicate the chamber in which the response to sensing is provided.

Intervals defined by the pacer timing and control module may include atrial and ventricular pacing escape intervals, refractory periods during which sensed P-waves and R-waves are ineffective to restart timing of the escape intervals, and the pulse widths of the pacing pulses. As another example, the pace timing and control module may define a blanking time period and provide signals from sensing module 86 to blank one or more channels, e.g., amplifiers, for a period during and after delivery of electrical stimulation to the heart 12. The durations of these intervals may be determined by the computing apparatus 80 in response to stored data in the memory 82. The pacer timing and control module may also determine the amplitude of the cardiac pacing pulses.

During pacing, escape interval counters within the pacer timing/control module may be reset upon sensing of R-waves and P-waves. The stimulation generator 84 may include pacer output circuits that are coupled, e.g., selectively by a switching module, to any combination of the electrodes 44, 48, 50, 58, 66, or 75 appropriate for delivery of a bipolar or unipolar pacing pulse to one of the chambers of the heart 12. The computing apparatus 80 may reset the escape interval counters upon the generation of pacing pulses by stimulation generator 84, and thereby control the basic timing of cardiac pacing functions, including anti-tachyarrhythmia pacing.

In some examples, the computing apparatus 80 may operate as an interrupt driven device and is responsive to interrupts from pacer timing and control module, where the interrupts may correspond to the occurrences of sensed P-waves and R-waves and the generation of cardiac pacing pulses. Any necessary mathematical calculations to be performed by the computing apparatus 80 and any updating of the values or intervals controlled by the pacer timing and control module of the computing apparatus 80 may take place following such interrupts. A portion of the memory 82 may be configured as a plurality of recirculating buffers, capable of holding series of measured intervals, which may be analyzed by the computing apparatus 80 in response to the occurrence of a pace or sense interrupt to determine whether the patient's heart 12 is presently exhibiting atrial or ventricular tachyarrhythmia.

Each of cardiac conduction system capture, injury of current, and impedance may be monitored by the computing apparatus 80, 81, 83 during implantation of the implantable electrode. The cardiac conduction system capture may be determined based on EGM or ECG signal analysis (e.g., based on left ventricular activation time, maximum rates of change following a pacing pulse, etc.). The injury of current may be determined based on EGM or ECG signal analysis (e.g., based on the “ST” segment, or the interval between ventricular depolarization and repolarization, etc.). The impedance may be determined based on EGM or ECG signal analysis (e.g., based on measured potential difference between electrodes, etc.). The computing apparatus 81 may determine the position of the implantable electrode using EGM and ECG signal analysis of cardiac conduction system capture, injury of current, and impedance.

The telemetry module 88 includes any suitable hardware, firmware, software, or any combination thereof for communicating with another device, such as the programmer 24 and/or the external electrode apparatus 45. Under the control of the computing apparatus 80, the telemetry module 88 may receive downlink telemetry from and send uplink telemetry to the programmer 24 with the aid of an antenna, which may be internal and/or external. The computing apparatus 80 may provide the data to be uplinked to the programmer 24 and the control signals for the telemetry circuit within the telemetry module 88, e.g., via an address/data bus. In some examples, the telemetry module 88 may provide received data to the computing apparatus 80 via a multiplexer. In some embodiments, the programmer 24 and/or external electrode apparatus 45 include a similar telemetry module to the telemetry module 88 as described herein.

The various components of the IMD 16 are coupled to the power source 90, which may include a rechargeable or non-rechargeable battery. A non-rechargeable battery may be selected to last for several years, while a rechargeable battery may be inductively charged from an external device, e.g., on a daily or weekly basis. In some embodiments, the programmer 24 and/or external electrode apparatus 45 include a similar power source to the power source 90 as described herein.

The illustrative systems, devices, and methods described herein may provide an effective and efficient way to implant an electrode into a patient proximate a portion of the cardiac conduction system (e.g., the LBB) using an implantable electrode (e.g., electrodes 48, 50). The implantable electrode may include one or more implantable electrodes, as described herein. The illustrative systems, devices, and methods described herein may provide and use monitored electrical activity (e.g., using internal and/or external electrodes to obtain EGM and/or ECG signals, respectively) to determine cardiac conduction system capture, injury of current, and impedance, and to further determine, during implantation of the electrode(s), that the electrode(s) are implanted proximate the portion of the cardiac conduction system based on the cardiac conduction system capture, injury of current, and impedance. The monitoring of electrical activity may be used to help decrease the incidence of perforation of the interventricular septal wall during advancement of the electrode(s) during implantation of the electrode(s) (e.g., in real time).

In at least one embodiment, and as illustrated in FIGS. 4-7, therapy system 71 may be used in assisting implantation of the implantable electrode(s) (e.g., electrodes 48, 50). The therapy system 71 may include an implantable lead (e.g., the cardiac conduction system pacing therapy lead 18) having the one or more implantable electrodes 48, 50. In alternative embodiments, a leadless pacing devices may be used as described herein.

Throughout this application, an implantable electrode may be described, which may be understood as one or both of implantable electrodes 48, 50. The implantable electrodes 48, 50 may be configured to deliver cardiac conduction system pacing proximate a portion of a patient's cardiac conduction system. The implantable electrodes 48, 50 may be configured to deliver cardiac conduction system pacing proximate a portion of the patient's LBB. The cardiac conduction system pacing may include LBB pacing or LBB area pacing. LBB pacing may be defined as pacing of the LBB directly, and LBB area pacing may be defined as pacing near or at the LBB. In alternative embodiments, other portions of the cardiac conduction system may be paced (e.g., RBB, His bundle, Purkinje fibers, etc.). The implantable electrodes 48, 50 may be further configured to sense electrical activity of the patient's heart 12.

As illustrated in FIG. 3, the external electrode apparatus 45 (including the external electrode(s) 44 and processing components 83) may be in wired or wireless communication with the IMD 16, and may additionally or alternatively be in wired or wireless communication with the programmer 24. The external electrode 44 may be configured to at least sense electrical activity of the patient's heart 12, as described herein. The programmer 24 (including the display 47 and processing components 81) may be in wired or wireless communication with the IMD 16, and may additionally or alternatively be in wired or wireless communication with the external electrode apparatus 45. The display 47 may include, for example, the Medtronic SMARTSYNC™ portable display. The use of the trademark SMARTSYNC™ has been noted in this application.

As described herein, the computing apparatus 80, 81, 83 may include processing circuitry, and may be operably coupled to the implantable electrodes 48, 50 and the external electrode(s) 44. The computing apparatus 81, 83 may be configured to monitor internal electrical activity using the implantable electrodes 48, 50 during implantation of the implantable electrode(s). The computing apparatus 81, 83 may be further configured to monitor external electrical activity using the external electrode(s) 44 during implantation of the implantable electrode(s).

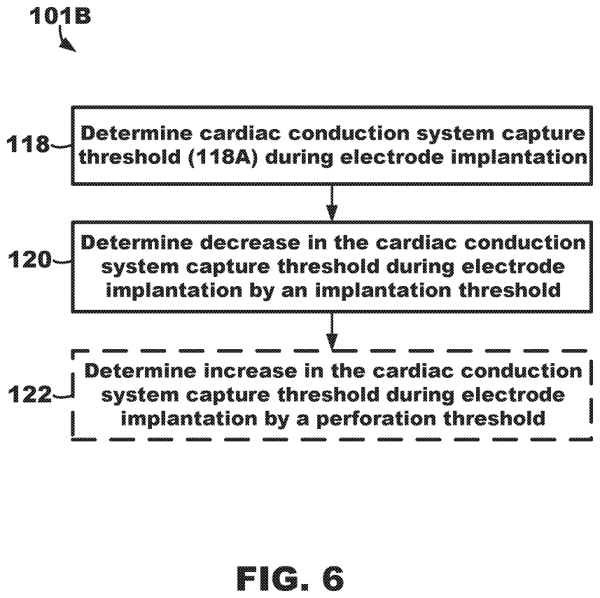

The computing apparatus 81 may be further configured to determine cardiac conduction system capture 117A (shown in FIG. 4) based on at least one of the internal and external monitored electrical activity during implantation of the implantable electrode(s). Cardiac conduction system capture 117A may be defined as successful delivery of intended cardiac conduction system pacing (as opposed to pacing, for example, myocardial tissue). A cardiac conduction system capture threshold 118A (shown in FIG. 6) may be used to define a minimum amount of power utilized to deliver the cardiac conduction system pacing while achieving capture of the cardiac conduction system 117A and may be measured in volts. The cardiac conduction system capture threshold 118A may be monitored in real time during implantation of the implantable electrode(s) using at least one of the implantable electrodes 48, 50 and the external electrode 44.

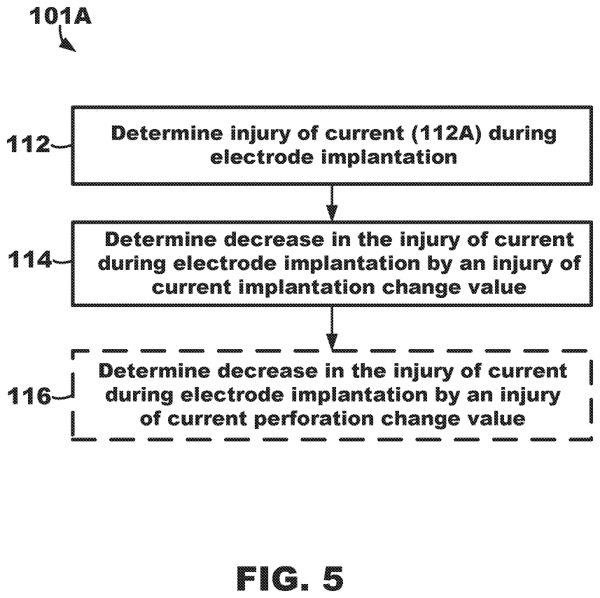

The computing apparatus 81 may be further configured to determine injury of current 112A (shown in FIGS. 4, 5) based on at least one of the monitored internal and external electrical activity during implantation of the implantable electrode(s). Injury of current 112A may be defined as the electrical current generated when an injured part of a nerve (e.g., a nerve bundle), muscle, or other excitable tissue is connected through a conductor with an uninjured region. The injured tissue will have a negative voltage compared to the uninjured tissue. During implantation, the implantable electrode(s) themselves may create minor tissue injury, and certain measured levels of injury of current may correspond to more optimal electrode placement. If a lead is being used, the lead itself may create minor tissue injury, and certain measured levels of injury of current may correspond to more optimal lead placement. Injury of current 112A may be measured in millivolts. Injury of current 112A may be monitored in real time during implantation of the implantable electrode(s) using at least one of the implantable electrodes 48, 50 and the external electrode 44.

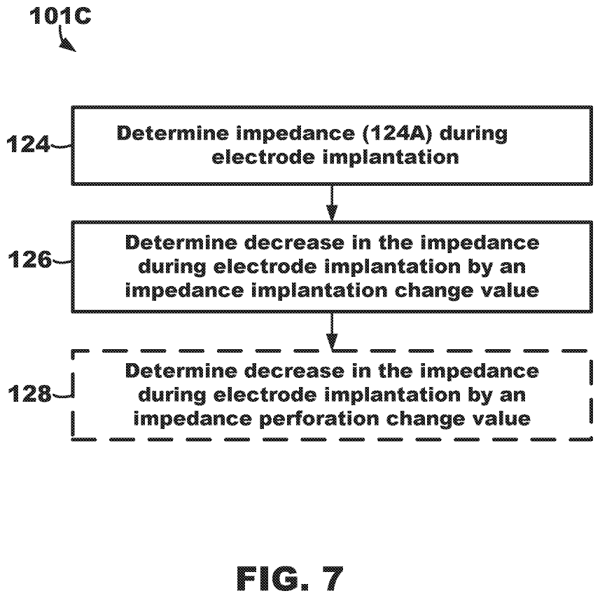

The computing apparatus 81 may be further configured to determine impedance 124A (shown in FIGS. 4, 7) based on at least one of the monitored internal and external electrical activity during implantation of the implantable electrode(s). Impedance 124A may be defined as the effective resistance to pacing current arising from the combined effective of resistance and reactance in a circuit. In other words, impedance may be defined as opposition to electrical flow, and may be measured in ohms. Impedance 124A may be monitored in real time during implantation of the implantable electrode(s) using at least one of the implantable electrodes 48, 50 and the external electrode 44.

The computing apparatus 81 may be further configured to determine, during implantation of the implantable electrode(s), that the implantable electrode(s) are implanted in the interventricular septal wall 35 proximate the cardiac conduction system and prior to perforation into the LV chamber 32 based on at least one of the cardiac conduction system capture 117A, the injury of current 112A, and the impedance 124A. Such determination is discussed further herein. Determining that the implantable electrode(s) are implanted in the interventricular septal wall 35 proximate the cardiac conduction system and prior to perforation into the LV chamber 32 based on at least one of the cardiac conduction system capture 117A, the injury of current 112A, and the impedance 124A may include determining that the implantable electrode(s) are implanted in the interventricular septal wall 35 proximate the LBB 8a. Such implantation proximate the LBB may allow for LBB pacing and/or LBB area pacing.

The computing apparatus 81 may be further configured to issue, or initiate, a first notification 110 in response to determining that the implantable electrode(s) are implanted in the interventricular septal wall 35 proximate the cardiac conduction system and prior to perforation of the LV chamber 32. The first notification 110 may be audible, visual, or in any other format to notify a user who is implanting the implantable electrode(s) that the implantable electrode(s) are implanted proximate the cardiac conduction system. The first notification 110 may be displayed (audibly, visually, etc.) on or by the display 47 as described herein.

The computing apparatus 81 may be further configured to determine that the implantable electrode(s) perforated into the LV chamber 32 based on at least one of the cardiac conduction system capture threshold 118A, the injury of current 112A, and the impedance 124A. The computing apparatus 81 may be further configured to issue a second notification 111 in response to determining that the implantable electrode(s) perforated into the LV chamber 32. The second notification 111 may be audible, visual, or in any other format to notify a user who is implanting the implantable electrode(s) that the implantable electrode(s) have perforated into the LV chamber 32. The second notification 111 may be displayed (audibly, visually, etc.) on or by the display 47 as described herein.

The computing apparatus 81 may be further configured to display the first notification 110 during implantation of the implantable electrode(s), e.g. using the display 47. The computing apparatus 81 may be further configured to display the second notification 111 during implantation of the implantable electrode(s), e.g. using the display 47.

In at least one embodiment, and as illustrated in FIGS. 4-7, the therapy system 71 may perform a method 100 to assist in implanting the implantable electrode(s) as described herein. The method 100 may include monitoring internal electrical activity using the one or more of the implantable electrodes 48, 50 during implantation of the implantable electrode(s) 102. The method 100 may further include monitoring external electrical activity using the one or more external electrode(s) 44 during implantation of the implantable electrode(s) 104. Monitoring of the internal and external electrical activity may include monitoring an EGM signal produced by the implantable electrodes 48, 50 and/or monitoring an ECG signal produced by the external electrode(s) 44.

The method 100 may further include determining cardiac conduction system capture 117A based on at least one of the internal and external monitored electrical activity during implantation of the implantable electrode(s) 106. The method 100 may further include determining injury of current 112A based on at least one of the monitored internal and external electrical activity during implantation of the implantable electrode(s) 106. The method 100 may further include determining impedance 124A based on at least one of the monitored internal and external electrical activity during implantation of the implantable electrode(s) 106.

The method 100 may further include determining, during implantation of the implantable electrode(s), that the implantable electrode(s) are implanted in the interventricular septal wall 35 proximate the cardiac conduction system and prior to perforation into the LV chamber 32 based on at least one of the cardiac conduction system capture 117A, the injury of current 112A, and the impedance 124A (altogether noted as reference numeral 108 in FIG. 4), as described further herein. The method 100 may further include issuing the first notification 110 in response to determining that the implantable electrode(s) are implanted in the interventricular septal wall 35 proximate the cardiac conduction system and prior to perforation of the LV chamber 32 (such issuance noted as reference number 110 in FIG. 4). The method 100 may further include determining that the implantable electrode(s) perforated into the LV chamber 32 based on at least one of the cardiac conduction system capture threshold 118A, the injury of current 112A, and the impedance 124A, as described further herein. The method 100 may further include issuing the second notification 111 in response to determining that the implantable electrode(s) perforated into the LV chamber 32 (such issuance noted as reference number 111 in FIG. 4, and further as an optional step to method 100).

A method 101A for determining a change in injury of current 112A during electrode(s) implantation for use in method 100 is illustrated in FIG. 5. Determining that the implantable electrode(s) are implanted in the interventricular septal wall 35 proximate the cardiac conduction system and prior to perforation into the LV chamber 32 based on the injury of current 112A may include determining the injury of current during electrode implantation 112. Determination of the injury of current during electrode implantation 112 may include determining the injury of current based on at least one of EGM or ECG signal analysis (e.g., based on the “ST” segment, understood as the interval between ventricular depolarization and repolarization, the amplitude of the signal, the amplitude of the ST segment of the signal, the amplitude of the ST segment relative to the R-wave of the signal, etc., and any combination thereof). The signal analysis may be based on the signal obtained from at least one of the one or more implantable electrodes 48, 50 and the external electrode(s) 44. The computing apparatus 81 may determine the position of the implantable electrode(s) using the monitored electrical activity (e.g., ECG and EGM signals) of the implantable electrode(s) to determine the injury of current.

The method 101A may further include determining that the implantable electrode(s) are implanted in the interventricular septal wall 35 proximate the cardiac conduction system and prior to perforation into the LV chamber 32 in response to determining a decrease in monitored injury of current 112A while the implantable electrode is moved through the ventricular septum 35 towards the LV chamber 32 during implantation of the implantable electrode(s). Determining the decrease in the monitored injury of current 112A may include determining the decrease in the monitored injury of current 112A by an injury of current (IOC) implantation change value 114.

The IOC implantation change value 114 may be between about 0 mV and about 20 mV. In at least one embodiment, the IOC implantation change value 114 may be greater than 0 mV. In other embodiments, the IOC implantation change value 114 may be greater than or equal to 0.1 mV, greater than or equal to 1 mV, greater than or equal to 2 mV, greater than or equal to 3 mV, greater than or equal to 4 mV, greater than or equal to 5 mV, greater than or equal to 6 mV, etc. and/or less than or equal to 20 mV, 6.5 mV, 5.5 mV, 4.5 mV, 3.5 mV, 2.5 mV, 1.5 mV, 0.5 mV, etc. In alternative embodiments, the IOC implantation change value 114 may be a set percentage of the patient's injury of current monitored at or near the ventricular septum.

Thus, if the IOC implant change value 114 is 0.1 mV, and an IOC decrease of 0.2 mV is monitored 112, then it may be determined an IOC decrease has occurred to indicate that the implantable electrode(s) are implanted in the interventricular septal wall 35 proximate the cardiac conduction system and prior to perforation into the LV chamber 32. If the IOC implant change value 114 is 5 mV, and an IOC decrease of 2 mV is monitored 112, then it may be determined an IOC decrease has not occurred to indicate that the implantable electrode(s) are not implanted in the interventricular septal wall 35 proximate the cardiac conduction system and prior to perforation into the LV chamber 32.

In alternative embodiments, determining the decrease in the monitored injury of current 112A may include determining the decrease in the monitored injury of current 112A to an IOC implantation target value. The IOC implantation target value may be between about 0 mV and about 5 mV. In at least one embodiment, the IOC implantation target value may be about equal to or greater than 2 mV. In other embodiments, the IOC implantation target value may be greater than 0 m V and/or greater than or equal to 0.1 mV, greater than or equal to 1 mV, greater than or equal to 2 mV, greater than or equal to 3 mV, greater than or equal to 4 mV, greater than or equal to 5 mV, etc. and/or less than or equal to 5.5 mV, 4.5 mV, 3.5 mV, 2.5 mV, 1.5 mV, 0.5 mV, etc. In alternative embodiments, the IOC implantation target value may be a set percentage of the patient's injury of current monitored at or near the ventricular septum.

Thus, if the IOC implantation target value is 2 mV, and an IOC decrease to 2 mV is monitored 112, then it may be determined an IOC decrease has occurred to indicate that the implantable electrode(s) are implanted in the interventricular septal wall 35 proximate the cardiac conduction system and prior to perforation into the LV chamber 32. If the IOC implantation target value is 2 mV, and an IOC decrease to 2.5 mV is monitored 112, then it may be determined an IOC decrease has not occurred to indicate that the implantable electrode(s) are not implanted in the interventricular septal wall 35 proximate the cardiac conduction system and prior to perforation into the LV chamber 32.

The method 101A may further include determining that the implantable electrode(s) have perforated into the LV chamber 32 based on the injury of current 112A. Such determination may include determining that the implantable electrode(s) have perforation into the LV chamber 32 in response to determining a further decrease in monitored injury of current 112A while the implantable electrode(s) 48, 50 are moved through the ventricular septum 35 from an implantation position proximate the cardiac conduction system into the LV chamber 32 during implantation of the implantable electrode(s). Determining the further decrease in monitored injury of current 112A may include determining the further decrease in monitored injury of current 112A by an IOC perforation change value 116. As discussed herein, the “further decrease” in the monitored injury of current is relative to the injury of current value monitored at or near the cardiac conduction system, or relative to the IOC value monitored at a location understood as at or near the cardiac conduction system.

The IOC perforation change value 116 may be between about 0.1 mV and about 20 mV. In at least one embodiment, the IOC perforation change value 116 may be greater than 1 mV. In other embodiments, the IOC perforation change value 116 may be greater than 0 mV and/or greater than or equal to 1 mV, greater than or equal to 2 mV, greater than or equal to 3 mV, greater than or equal to 4 mV, greater than or equal to 5 mV, greater than or equal to 10 mV, etc. and/or less than or equal to 20.5 mV, 6.5 mV, 5.5 mV, 4.5 mV, 3.5 mV, 2.5 mV, 1.5 mV, etc. In alternative embodiments, the IOC perforation change value 116 may be a set percentage of the patient's IOC implantation change value 114, or may be a set percentage of the patient's injury of current monitored at or near the cardiac conduction system.

Thus, if the IOC perforation change value 116 is 1 mV, and a further IOC decrease of 1.2 mV is monitored 112, then it may be determined a further IOC decrease has occurred to indicate that the implantable electrode(s) have perforated into the LV chamber 32. If the IOC perforation change value 116 is 1 mV, and a further IOC decrease of 0.7 mV is monitored 112, then it may be determined a further IOC decrease has not occurred to indicate that the implantable electrode(s) have not perforated into the LV chamber 32.

In alternative embodiments, determining the further decrease in the monitored injury of current 112A may include determining the further decrease in the monitored injury of current 112A to an IOC perforation target value. In further alternative embodiments, if the monitored injury of current 112A disappears, or is no longer monitorable, such disappearance may be equated to the further decrease in the monitored injury of current 112A to an IOC perforation target value.

The IOC perforation target value may be between about 0 mV and about 2 mV. In at least one embodiment, the IOC perforation target value may be about equal to or greater than 1 mV. In other embodiments, the IOC perforation target value may be greater than 0 mV and/or greater than or equal to 2 mV, greater than or equal to 1 mV, greater than or equal to 0.5 mV, greater than or equal to 0.25 mV, greater than or equal to 0.2 mV, greater than or equal to 0.1 mV, greater than or equal to 0 mV, etc. and/or less than or equal to 0.3 mV, 0.6 mV, 0.9 mV, 1.1 mV, 1.6 mV, 1.9 mV, etc. In alternative embodiments, the IOC perforation target value may be a set percentage of the patient's IOC implantation target value, or may be a set percentage of the patient's injury of current monitored at or near the cardiac conduction system.

Thus, if the IOC perforation target value is 1 mV, and an IOC decrease to 1 mV is monitored 112, then it may be determined an IOC decrease has occurred to indicate that the implantable electrode(s) have perforated into the LV chamber 32. If the IOC perforation target value is 1 mV, and an IOC decrease to 1.5 mV is monitored 112, then it may be determined an IOC decrease has not occurred to indicate that the implantable electrode(s) have perforated into the LV chamber 32.

A method 101B for determining the cardiac conduction system capture threshold 118A during electrode(s) implantation for use in method 100 is illustrated in FIG. 6. As discussed herein, the cardiac conduction system capture threshold 118A may be a minimum amount of power (e.g., voltage) utilized to deliver the cardiac conduction system pacing while achieving capture of the cardiac conduction system 117A (shown in FIG. 4). Thus, capture of the cardiac conduction system 117A may be established prior to determining the cardiac conduction system capture threshold 118A. Illustrative establishment of cardiac conduction system capture may be described in, for example, U.S. Pat. No. 11,007,369 B2 entitled “Implantable medical device and method for determining His bundle pacing capture” published on May 9, 2019 and granted on May 18, 2021, which is incorporated herein by reference in its entirety, and in U.S. Pat. App. No. 2022/0080210 A1 entitled “His-purkinje system capture detection” published on Mar. 17, 2022, which is incorporated herein by reference in its entirety.

Determining that the implantable electrode(s) are implanted in the interventricular septal wall 35 proximate the cardiac conduction system and prior to perforation into the LV chamber 32 based on the cardiac conduction system capture threshold 118A may include determining the cardiac conduction system capture threshold during electrode implantation 118. Determination of the cardiac conduction system capture threshold during electrode implantation 118 may include determining the cardiac conduction system capture threshold 118A based on at least one of EGM or ECG signal analysis (e.g., based on left ventricular activation time, maximum rates of change following a pacing pulse, specific QRS morphology, etc., and any combination thereof). Illustrative determination of cardiac conduction system capture may be described in, for example, U.S. Pat. App. Pub. No. 2020/0306546 A1 entitled “Cardiac Conduction System Capture” published on Oct. 1, 2020, which is incorporated herein by reference in its entirety. The signal analysis may be based on the signal obtained from at least one of the one or more implantable electrodes 48, 50 and the external electrode(s) 44. The computing apparatus 81 may determine the position of the implantable electrode(s) using the monitored electrical activity (e.g., ECG and EGM signals) of the implantable electrode(s) to determine the cardiac conduction system capture threshold 118A.

The method 101B may further include determining that the implantable electrode(s) are implanted in the interventricular septal wall 35 proximate the cardiac conduction system and prior to perforation into the LV chamber 32 in response to determining an increase in the cardiac conduction system capture threshold 118A while the implantable electrode is moved through the ventricular septum 35 towards the LV chamber 32 during implantation of the implantable electrode(s).

Determining the increase in the cardiac conduction system capture threshold 118A may include a determination that the cardiac conduction system capture threshold 118A has increased by an implantation threshold 120. The implantation threshold 120 may be between about 0 V and about 2 V. In at least one embodiment, the implantation threshold 120 may be about equal to or greater than 1 V. In other embodiments, the implantation threshold 120 may be less than or equal to 2 V, less than or equal to 1.75 V, less than or equal to 1.5 V, less than or equal to 1 V, less than or equal to 0.75 V, less than or equal to 0.5 V, less than or equal to 0.2 V, etc. and/or greater than 0 V and/or greater than or equal to 0.1 V, 0.6 V, 0.9 V, 1.1 V, 1.6 V, 1.9 V, 2.1 V, etc. In another embodiment, the implantation threshold 120 may be a set percentage of the patient's intrinsic cardiac conduction system capture threshold 118A.

Thus, if the implantation threshold 120 is 1 V, and a conduction system capture threshold increase of 1.2 V is monitored 118, then it may be determined that a conduction system capture threshold increase has occurred to indicate that the implantable electrode(s) are implanted in the interventricular septal wall 35 proximate the cardiac conduction system and prior to perforation into the LV chamber 32. If the implantation threshold 120 is 1 V, and a conduction system capture threshold increase of 0.8 V is monitored 118, then it may be determined that a conduction system capture threshold increase has not occurred to indicate that the implantable electrode(s) are not implanted in the interventricular septal wall 35 proximate the cardiac conduction system and prior to perforation into the LV chamber 32.

In an alternative embodiment, the method 101B may further include determining that the implantable electrode(s) are implanted in the interventricular septal wall 35 proximate the cardiac conduction system and prior to perforation into the LV chamber 32 in response to determining a decrease in the cardiac conduction system capture threshold 118A while the implantable electrode is moved through the ventricular septum 35 towards the LV chamber 32 during implantation of the implantable electrode(s).

Determining the decrease in the cardiac conduction system capture threshold 118A may include a determination that the cardiac conduction system capture threshold 118A has decreased by an initial implantation threshold. The initial implantation threshold may be between about 0 V and about 2 V. In at least one embodiment, the initial implantation threshold may be about equal to or greater than 1 V. In other embodiments, the initial implantation threshold may be less than or equal to 2 V, less than or equal to 1.75 V, less than or equal to 1.5 V, less than or equal to 1 V, less than or equal to 0.75 V, less than or equal to 0.5 V, less than or equal to 0.2 V, etc. and/or greater than 0 V and/or or greater than or equal to 0.1 V, 0.6 V, 0.9 V, 1.1 V, 1.6 V, 1.9 V, 2.1 V, etc. In another embodiment, the initial implantation threshold may be a set percentage of the patient's intrinsic cardiac conduction system capture threshold 118A.

Thus, if the initial implantation threshold is 1 V, and a conduction system capture threshold decrease of 1.2 V is monitored 118, then it may be determined that a conduction system capture threshold decrease has occurred to indicate that the implantable electrode(s) are implanted in the interventricular septal wall 35 proximate the cardiac conduction system and prior to perforation into the LV chamber 32. If the initial implantation threshold is 1 V, and a conduction system capture threshold decrease of 0.8 V is monitored 118, then it may be determined that a conduction system capture threshold decrease has not occurred to indicate that the implantable electrode(s) are not implanted in the interventricular septal wall 35 proximate the cardiac conduction system and prior to perforation into the LV chamber 32.

The method 101B may further include determining that the implantable electrode(s) have perforated into the LV chamber 32 based on the cardiac conduction system capture threshold 118A. Such determination may include determining that the implantable electrode(s) have perforated into the LV chamber 32 in response to determining a further increase in the cardiac conduction system capture threshold 118A while the implantable electrode(s) 48, 50 are moved through the ventricular septum 35 from an implantation position proximate the cardiac conduction system into the LV chamber 32 during implantation of the implantable electrode(s). Determining the further increase in the cardiac conduction system capture threshold 118A may include determining the cardiac conduction system capture threshold 118A has further increased by a perforation threshold 122. As discussed herein, “further” is relative to the capture threshold value determined at or near the cardiac conduction system, or the capture threshold value determined at a location understood as at or near the cardiac conduction system.

The perforation threshold 122 may be between about 0.1 V and about 5 V. In at least one embodiment, the perforation threshold 122 may be about equal to or greater than 1 V. In other embodiments, the perforation threshold 122 may be greater than 0 V, greater than or equal to 0.1 V, greater than or equal to 0.15 V, greater than or equal to 0.5 V, greater than or equal to 1 V, greater than or equal to 2 V, greater than or equal to 5 V, etc. and/or less than or equal to 4.5 V, 3.5 V, 2.5 V, 1.5 V, 0.4 V, 0.2 V, 0.1 V, etc. In alternative embodiments, the perforation threshold 122 may be a set percentage of the patient's implantation threshold 120, or may be a set percentage of the patient's intrinsic cardiac conduction system capture threshold 118A, or may be a set percentage of the patient's cardiac conduction system capture threshold as determined at or near the cardiac conduction system.

Thus, if the perforation threshold 122 is 1 V, and a conduction system capture threshold increase of 1.1 V is monitored 118, then it may be determined that a further conduction system capture threshold increase has occurred to indicate that the implantable electrode(s) have perforated into the LV chamber 32. If the perforation threshold 122 is 1 V, and a conduction system capture threshold increase of 0.9 V is monitored 118, then it may be determined that a further conduction system capture threshold increase has not occurred to indicate that the implantable electrode(s) have not perforated into the LV chamber 32.

In alternative embodiments, determining the further increase in the cardiac conduction system capture threshold 118A may include determining the cardiac conduction system capture threshold 118A has further increased at least to a capture perforation target value. In further alternative embodiments, if the cardiac conduction system capture threshold 118A disappears, or the cardiac conduction system is no longer captured at all, such loss of capture may be equated to the cardiac conduction system capture threshold 118A reaching the perforation target value.

Determining the further increase in the cardiac conduction system capture threshold 118A may include a determination that the cardiac conduction system capture threshold 118A has further increased to a capture perforation target value. The capture perforation target value may be between about 0.1 V and about 5.0 V. In at least one embodiment, the capture perforation target value may be about equal to or greater than 1.0 V. In other embodiments, the capture perforation target value may be less than or equal to 5 V, less than or equal to 4.0 V, less than or equal to 3.0 V, less than or equal to 2.0 V, less than or equal to 1.0 V, less than or equal to 0.5 V, less than or equal to 0.4 V, less than or equal to 0.3 V, less than or equal to 0.2 V, less than or equal to 0.1 V, etc. and/or greater than 0 V, greater than or equal to 0.15 V, 0.25 V, 0.35 V, 0.45 V, 0.55 V, 0.65 V, 0.75 V, 0.85 V, 0.95 V, 1.05 V, 1.5 V, 2.5 V, 3.5 V, 4.5 V, etc. In another embodiment, the capture perforation target value may be a set percentage of the patient's intrinsic cardiac conduction system capture threshold 118A, or may be a set percentage of the implantation target value, or may be a set percentage the patient's cardiac conduction system capture threshold as determined at or near the cardiac conduction system.

Thus, if the capture perforation target value is 1.0 V, and a conduction system capture threshold increase to 1.1 V is monitored 118, then it may be determined that a conduction system capture threshold increase has occurred to indicate that the implantable electrode(s) have perforated into the LV chamber 32. If the capture perforation target value is 1.0 V, and a conduction system capture threshold increase to 0.5 V is monitored 118, then it may be determined that a conduction system capture threshold increase has not occurred to indicate that that the implantable electrode(s) have perforated into the LV chamber 32.

A method 101C for determining the impedance 124A during electrode(s) implantation for use in method 100 is illustrated in FIG. 7. Determining that the implantable electrode(s) are implanted in the interventricular septal wall 35 proximate the cardiac conduction system and prior to perforation into the LV chamber 32 based on the impedance 124A may include determining the impedance during electrode implantation 124. Determination of the impedance during electrode implantation 124 may include determining the impedance based on at least one of EGM or ECG signal analysis (e.g., based on measured potential difference between implanted electrode(s) and the pulse generator in a unipolar configuration as discussed herein, based on the measured potential difference between the implanted electrodes in a bipolar configuration as discussed herein, etc.). The signal analysis may be based on the signal obtained from at least one of the one or more implantable electrodes 48, 50 and the external electrode(s) 44. The computing apparatus 81 may determine the position of the implantable electrode(s) using the monitored electrical activity (e.g., ECG and EGM signals) of the implantable electrode(s) to determine the impedance.

The method 101C may further include determining that the implantable electrode(s) are implanted in the interventricular septal wall 35 proximate the cardiac conduction system and prior to perforation into the LV chamber 32 based on the impedance in response to determination of a decrease in the monitored impedance 124A while the implantable electrode is moved through the ventricular septum 35 towards the LV chamber 32 during implantation of the implantable electrode(s).

Determining the decrease in the monitored impedance 124A may include a determination that the decrease in the monitored impedance 124A is by an impedance implantation change value 126. The impedance implantation change value 126 may be between about 10 ohms and about 200 ohms. In at least one embodiment, the impedance implantation change value 126 may be about equal to or greater than 100 ohms. In other embodiments, the impedance implantation change value 126 may be less than or equal to 200 ohms, less than or equal to 175 ohms, less than or equal to 150 ohms, less than or equal to 125 ohms, less than or equal to 100 ohms, less than or equal to 75 ohms, less than or equal to 50 ohms, etc. and/or greater than 0 ohms, greater than or equal to 10 ohms, 25 ohms, 80 ohms, 110 ohms, 125 ohms, 145 ohms, 180 ohms, 195 ohms, etc. In alternative embodiments, the impedance implantation change value 126 may be a set percentage of the patient's impedance monitored at or near the ventricular septum.

Thus, if the impedance implantation change value 126 is 100 ohms, and an impedance decrease of 125 ohms is monitored 124, then it may be determined an impedance decrease has occurred to indicate that the implantable electrode(s) are implanted in the interventricular septal wall 35 proximate the cardiac conduction system and prior to perforation into the LV chamber 32. If the impedance implantation change value 126 is 100 ohms, and an impedance decrease of 85 ohms is monitored 124, then it may be determined an impedance decrease has not occurred to indicate that the implantable electrode(s) are not implanted in the interventricular septal wall 35 proximate the cardiac conduction system and prior to perforation into the LV chamber 32.

The method 101C may further include determining that the implantable electrode(s) have perforated into the LV chamber 32 based on the determined impedance 124A. Such determination may include determining that the implantable electrode(s) have perforated into the LV chamber 32 in response to determining a further decrease in the monitored impedance 124A while the one or more implantable electrodes 48, 50 are moved through the ventricular septum 35 from an implantation position proximate the cardiac conduction system into the LV chamber 32 during implantation of the implantable electrode(s). Determining the further decrease in the monitored impedance 124A may include determining the impedance 124A has further decreased by an impedance perforation change value 128. As discussed herein, the “further decrease” in the monitored impedance is relative to the impedance monitored at or near the cardiac conduction system, or the impedance monitored at a location understood as at or near the cardiac conduction system.

The impedance perforation change value 128 may be between about 10 ohms and about 600 ohms. In at least one embodiment, the impedance perforation change value 128 may be about equal to or greater than 100 ohms. In other embodiments, the impedance perforation change value 128 may be greater than or equal to 10 ohms, 25 ohms, 50 ohms, 75 ohms, 150 ohms, 200 ohms, greater than or equal to 250 ohms, greater than or equal to 300 ohms, greater than or equal to 350 ohms, greater than or equal to 400 ohms, greater than or equal to 450 ohms, greater than or equal to 500 ohms, greater than or equal to 550 ohms, greater than or equal to 600 ohms, etc. and/or less than or equal to 590 ohms, 540 ohms, 490 ohms, 440 ohms, 390 ohms, 340 ohms, 290 ohms, 240 ohms, 190 ohms, 140 ohms, 30 ohms, etc. In another embodiment, the impedance perforation change value 128 may be a set percentage of the impedance implantation change value 126, or may be a set percentage of the patient's impedance monitored at or near the cardiac conduction system.

Thus, if the impedance perforation change value 128 is 100 ohms, and an impedance decrease of 115 ohms is monitored 124, then it may be determined a further impedance decrease has occurred to indicate that the implantable electrode(s) have perforated into the LV chamber 32. If the impedance perforation change value 128 is 100 ohms, and an impedance decrease of 190 ohms is monitored 124, then it may be determined a further impedance decrease has not occurred to indicate that the implantable electrode(s) have not perforated into the LV chamber 32.

During implantation of the implantable electrode(s), in some examples, if the injury of current 112A decreases by the IOC implantation change value 114 or decreases to the IOC implantation target value, and if the cardiac conduction system capture threshold 118A increases by the implantation threshold 120 or increases to the capture implantation target value, and if the impedance 124A decreases by the impedance implantation change value 126, then it may be determined that the implantable electrode(s) are implanted in the interventricular septal wall 35 proximate the LBB 8a and prior to perforation into the LV chamber 32. The first notification 110 may be issued in response to determining that the implantable electrode(s) are implanted in the interventricular septal wall 35 proximate the cardiac conduction system and prior to perforation of the LV chamber 32.

During continued advancement of the implantable electrode(s) from an implantation position proximate the cardiac conduction system within the ventricular septum 35, in some examples, if the injury of current 112A further decreases by the IOC perforation change value 116 or further decreases to the IOC perforation target value, and if the cardiac conduction system capture threshold 118A further increases by the perforation threshold 122 or further increases to the capture perforation target value, and if the impedance 124A further decreases by the impedance perforation change value 128, then it may be determined that the implantable electrode(s) have perforated into the LV chamber 32. The second notification 111 may be issued in response to determining that the implantable electrode(s) perforated into the LV chamber 32.

In some embodiments, all of cardiac conduction system capture, injury of current, and impedance are monitored during implantation of the implantable electrode, and all of cardiac conduction system capture, injury of current, and impedance are used to determine electrode position or perforation through the ventricular septum into an adjacent heart chamber. In alternative embodiments, not all of cardiac conduction system capture, injury of current, and impedance are monitored during implantation of the implantable electrode, and/or not all of cardiac conduction system capture, injury of current, and impedance are used to determine electrode position or perforation through the ventricular septum into an adjacent heart chamber. In such alternative embodiments, one or two of cardiac conduction system capture, injury of current, and impedance may be used to determine electrode position or perforation through the ventricular septum.

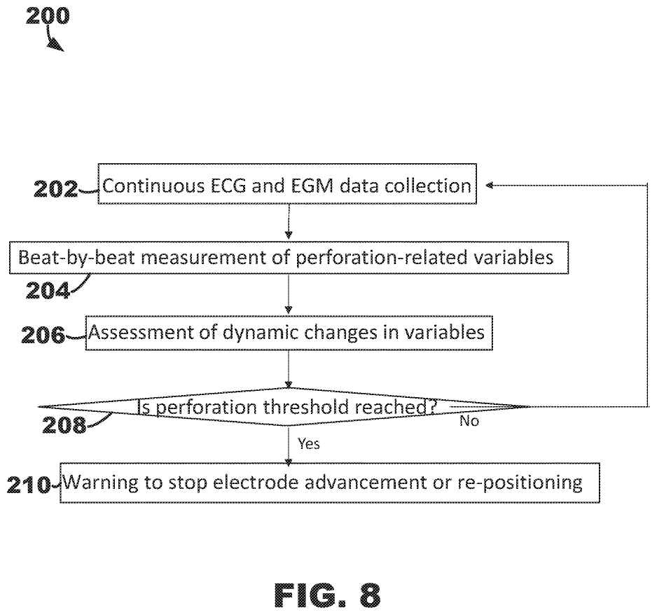

In at least one embodiment, and as illustrated in FIG. 8, the therapy system 71 may perform a method 200 to assist in implanting the implantable electrode(s) as described herein. The method 200 may include continuous ECG and EGM data collection 202 as beat-by-beat measurement of perforation-related variables (e.g., injury of current, cardiac conduction system capture, and impedance) 204. The method 200 may further include assessment of dynamic changes in the perforation-related variables 206. The method 200 may further include determining whether a perforation threshold has been reached based on the assessment of the dynamic changes in the perforation-related variables 208. If the perforation threshold is not reached, the method 200 may continue continuous ECG and EGM data collection 202. If the perforation threshold is reached, the method 200 may further include issuing a warning to stop advancement of the lead or to re-position the lead (noted with reference numeral 210).

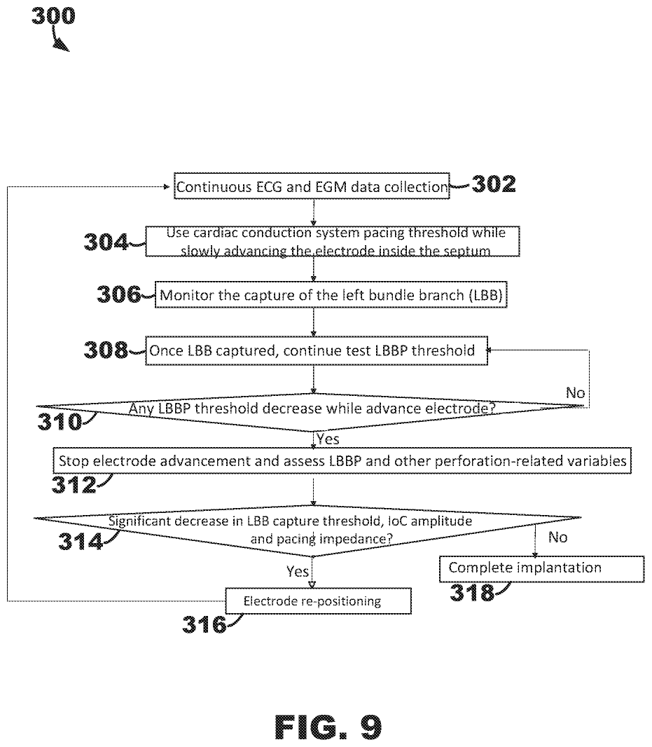

In at least one embodiment, and as illustrated in FIG. 9, the therapy system 71 may perform a method 300 to assist in implanting the implantable electrode(s) as described herein. The method 300 may include continuous ECG and EGM data collection 302 using a cardiac conduction system pacing threshold while slowly advancing the electrode(s) inside the intraventricular septal wall 35 (noted as reference numeral 304). The method 300 may further include monitoring the capture of the LBB 306. Once capture is established, the method 300 may continue continuous testing of the cardiac conduction system capture threshold (e.g., LBB cardiac conduction system capture threshold) 308.

The method 300 may further include determining whether the LBB cardiac conduction system capture threshold decreases while advancing the electrode 310. If the LBB cardiac conduction system capture threshold does not decrease during electrode(s) advancement, the method 300 may continue continuous testing of the LBB cardiac conduction system capture threshold (308). If the LBB cardiac conduction system capture threshold does decrease during electrode(s) advancement, then method 300 may further include stopping electrode(s) advancement and assessing the LBB cardiac conduction system capture threshold and other perforation-related variables (e.g., injury of current and impedance) 312. The method 300 may further include determining if a significant decrease in the LBB cardiac conduction system capture threshold (e.g. the threshold stops decreasing, increases, or the signal is lost), injury of current amplitude (e.g., the amplitude decreases significantly or the signal is lost), and pacing impedance (e.g., the impedance decreases significantly or the signal is lost) has occurred 314. If such significant decrease has not occurred, the method 300 may further include completion of electrode(s) implantation at or near the LBB 318. If such significant decrease has occurred, the method 300 may further include re-positioning of the electrode(s) (noted as reference numeral 316).

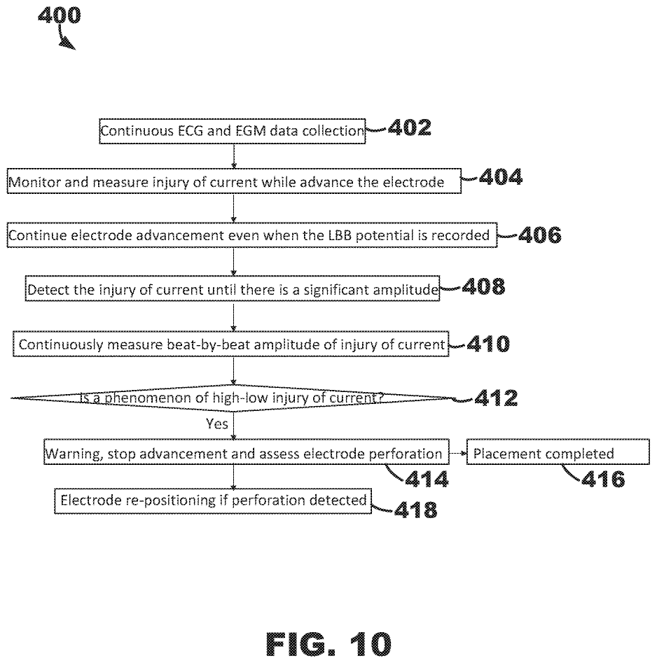

In at least one embodiment, and as illustrated in FIG. 10, the therapy system 71 may perform a method 400 to assist in implanting the implantable electrode(s) as described herein. The method 400 may include continuous ECG and EGM data collection 402, monitoring and measuring the injury of current while advancing the electrode(s) 404. The method 400 may further include continuing electrode(s) advancement even when LBB cardiac conduction system capture (noted as LBB potential in FIG. 10) is recorded 406.

The method 400 may further include detecting the injury of current until there is a significant injury of current amplitude 408 and continuously measuring beat-by-beat amplitude of the injury of current 410. The method 400 may further include determining if there is a phenomenon of high-to-low injury of current 412. If such high-to-low injury of current phenomenon does not occur, the method 400 may repeat and continue to continuously measure beat-by-beat amplitude of the injury of current 410. If such high-to-low injury of current phenomenon occurs, the method 400 may further include issuing a warning and stopping electrode(s) advancement and assessing potential electrode(s) perforation 414. The method 400 may further include completion of electrode(s) placement 416 if no perforation is detected. The method 400 may further include re-positioning the electrode(s) if perforation is detected 418.

Various examples have been described. These and other examples are within the scope of the following claims. For example, a single chamber, dual chamber, or triple chamber pacemakers (e.g., CRT-P) or ICDs (e.g., CRT-D), or leadless devices and changes in LBB (or conduction system) capture threshold, injury of current, pacing impedance, or other parameters that suggest tissue perforation can be used to implement the illustrative methods described herein.

### Illustrative Examples

While the present disclosure is not so limited, an appreciation of various aspects of the disclosure will be gained through a discussion of the specific illustrative examples provided below. Various modifications of the illustrative examples, as well as additional examples of the disclosure, will become apparent herein.
Example Ex1: A system for use in assisting implantation of an implantable electrode, the system comprising: an implantable electrode configured to deliver cardiac conduction system pacing proximate a portion of a patient's cardiac conduction system; an external electrode configured to at least sense electrical activity of the patient's heart; and a computing apparatus comprising processing circuitry, the computing apparatus operably coupled to the implantable electrode and the external electrode, wherein the computing apparatus is configured to, during implantation of the implantable electrode: monitor internal electrical activity using the implantable electrode during implantation of the implantable electrode, monitor external electrical activity using the external electrode during implantation of the implantable electrode, determine cardiac conduction system capture based on at least one of the internal and external monitored electrical activity during implantation of the implantable electrode, determine injury of current based on at least one of the monitored internal and external electrical activity during implantation of the implantable electrode, determine impedance based on at least one of the monitored internal and external electrical activity during implantation of the implantable electrode, issue a first notification in response to determining that the implantable electrode is implanted in the interventricular septal wall proximate the cardiac conduction system and prior to perforation into the left ventricular (LV) chamber based on the cardiac conduction system capture, the injury of current, and the impedance.
Example Ex2: A method to assist in implanting an implantable electrode comprising an implantable electrode proximate a patient's cardiac conduction system, the method comprising, during implantation of the implantable electrode: monitoring internal electrical activity using the implantable electrode during implantation of the implantable electrode, monitoring external electrical activity using an external electrode during implantation of the implantable electrode, determining cardiac conduction system capture based on at least one of the internal and external monitored electrical activity during implantation of the implantable electrode, determining injury of current based on at least one of the monitored internal and external electrical activity during implantation of the implantable electrode, determining impedance based on at least one of the monitored internal and external electrical activity during implantation of the implantable electrode, issuing a first notification in response to determining that the implantable electrode is implanted in the interventricular septal wall proximate the cardiac conduction system and prior to perforation into the left ventricular (LV) chamber based on the cardiac conduction system capture, the injury of current, and the impedance.
Example Ex3: The system as in Example Ex1 or the method as in Example Ex2, wherein issuing a first notification in response to determining that the implantable electrode is implanted in the interventricular septal wall proximate the cardiac conduction system and prior to perforation into the LV chamber based on the cardiac conduction system capture, the injury of current, and the impedance comprises determining that the implantable electrode is implanted in the interventricular septal wall proximate the left bundle branch (LBB) for LBB pacing or LBB area pacing.
Example Ex4: The system or method as in any one of Examples Ex1-3, wherein issuing a first notification in response to determining that the implantable electrode is implanted in the interventricular septal wall proximate the cardiac conduction system and prior to perforation into the LV chamber based on the injury of current comprises determining that the implantable electrode is implanted in the interventricular septal wall proximate the cardiac conduction system and prior to perforation into the LV chamber in response to determining a decrease in monitored injury of current while the implantable electrode is moved through the ventricular septum towards the LV chamber during implantation of the implantable electrode.
Example Ex5: The system or method as in Example Ex4, wherein determining the decrease in monitored injury of current comprises determining the decrease in monitored injury of current (IOC) by an IOC implantation change value, wherein the IOC implantation change value is greater than 0 mV.
Example Ex6: The system or method as in any one of Examples Ex1-5, wherein a cardiac conduction system capture threshold is a minimum amount of power utilized to deliver the cardiac conduction system pacing while achieving capture of the cardiac conduction system, wherein issuing a first notification in response to determining that the implantable electrode is implanted in the interventricular septal wall proximate the cardiac conduction system and prior to perforation into the LV chamber based on the monitored cardiac conduction system capture comprises determining that the implantable electrode is implanted in the interventricular septal wall proximate the cardiac conduction system and prior to perforation into the LV chamber in response to determination of an increase in the cardiac conduction system capture threshold while the implantable electrode is moved through the ventricular septum towards the LV chamber during implantation of the implantable electrode.
Example Ex7: The system or method as in Example Ex6, wherein determining the increase in the cardiac conduction system capture threshold comprises determination that the cardiac conduction system capture threshold has increased by an implantation threshold, wherein the implantation threshold is greater than or equal to 1.0 Volt.
Example Ex8: The system or method as in any one of Examples Ex1-7, wherein issuing a first notification in response to determining that the implantable electrode is implanted in the interventricular septal wall proximate the cardiac conduction system and prior to perforation into the LV chamber based on the impedance comprises determining that the implantable electrode is implanted in the interventricular septal wall proximate the cardiac conduction system and prior to perforation into the LV chamber in response to determining a decrease in monitored impedance while the implantable electrode is moved through the ventricular septum towards the LV chamber during implantation of the implantable electrode.
Example Ex9: The system or method as in Example Ex8, wherein determining the decrease in monitored impedance comprises determining the decrease in monitored impedance by an impedance implantation change value, wherein the impedance implantation change value is greater than or equal to 100 ohms.
Example Ex 10: The system as in Example Ex 1 or the method as in Example Ex2, wherein the computing apparatus is further configured to execute or the method further comprises, during implantation of the implantable electrode: issuing a second notification in response to determining that the implantable electrode perforated into the LV chamber based on the cardiac conduction system capture threshold, the injury of current, and the impedance.
Example Ex11: The system or method as in Example Ex10, wherein issuing a second notification in response to determining that the implantable electrode has perforated into the LV chamber based on the injury of current comprises determining that the implantable electrode has perforated into the LV chamber in response to determining a decrease in monitored injury of current while the implantable electrode is moved through the ventricular septum from an implantation position proximate the cardiac conduction system into the LV chamber during implantation of the implantable electrode.
Example Ex 12: The system or method as in Example Ex11, wherein determining the decrease in monitored injury of current comprises determining the decrease in monitored injury of current by an IOC perforation change value, wherein the IOC perforation change value is greater than or equal to 1 mV.
Example Ex13: The system or method as in Example Ex10, wherein a cardiac conduction system capture threshold is a minimum amount of power utilized to deliver the cardiac conduction system pacing while achieving capture of the cardiac conduction system, wherein issuing a second notification in response to determining that the implantable electrode has perforated into the LV chamber based on the monitored cardiac conduction system capture comprises determining that the implantable electrode has perforated into the LV chamber in response to determining an increase in the cardiac conduction system capture threshold while the implantable electrode is moved through the ventricular septum from an implantation position proximate the cardiac conduction system into the LV chamber during implantation of the implantable electrode.
Example Ex14: The system or method as in Example Ex13, wherein determining the increase in the cardiac conduction system capture threshold comprises determination that the cardiac conduction system capture threshold has increased by a perforation threshold, wherein the perforation threshold is greater than or equal to 1.0 Volt.
Example Ex15: The system or method as in Example Ex10, wherein issuing a second notification in response to determining that the implantable electrode has perforated into the LV chamber based on the impedance comprises determining that the implantable electrode has perforated into the LV chamber in response to determining a decrease in monitored impedance while the implantable electrode is moved through the ventricular septum from an implantation position proximate the cardiac conduction system into the LV chamber during implantation of the implantable electrode.
Example Ex16: The system or method as in Example Ex15, wherein determining the decrease in monitored impedance comprises determining the decrease in monitored impedance by an impedance perforation change value, wherein the impedance perforation change value is greater than or equal to 100 ohms.
Example Ex17: The system as in Example Ex1 or the method as in Example Ex2, wherein the computing apparatus is further configured to execute or the method further comprises: displaying the first notification during implantation of the implantable electrode using a display, wherein the computing apparatus is operatively couplable to the display.
Example Ex18: The system or method as in Example Ex10, further comprising a display, wherein the computing apparatus is further configured to execute or the method further comprises: displaying the first notification during implantation of the implantable electrode using a display; and displaying the second notification during implantation of the implantable electrode using the display, wherein the computing apparatus is operatively couplable to the display.
Example Ex19: A system for use in assisting implantation of an implantable electrode, the system comprising: an implantable electrode configured to deliver cardiac conduction system pacing proximate a portion of a patient's cardiac conduction system; and a computing apparatus comprising processing circuitry, the computing apparatus operably coupled to the implantable electrode, wherein the computing apparatus is configured to, during implantation of the implantable electrode: monitor internal electrical activity using the implantable electrode during implantation of the implantable electrode, determine cardiac conduction system capture, injury of current, and impedance based on the monitored internal electrical activity during implantation of the implantable electrode, and issue a first notification in response to determining that the implantable electrode is implanted in the interventricular septal wall proximate the cardiac conduction system and prior to perforation into the left ventricular (LV) chamber based on at least one of the determined cardiac conduction system capture, the injury of current, and the impedance.
Example Ex20: A method to assist in implanting an implantable electrode comprising an implantable electrode proximate a patient's cardiac conduction system, the method comprising, during implantation of the implantable electrode: monitoring internal electrical activity using the implantable electrode during implantation of the implantable electrode, determining cardiac conduction system capture, injury of current, and impedance, based the monitored internal electrical activity during implantation of the implantable electrode, and issuing a first notification in response to determining that the implantable electrode is implanted in the interventricular septal wall proximate the cardiac conduction system and prior to perforation into the left ventricular (LV) chamber based on at least one of the cardiac conduction system capture, the injury of current, and the impedance.
Example Ex21: The system as in Example Ex19 or the method as in Example Ex20, wherein issuing a first notification in response to determining that the implantable electrode is implanted in the interventricular septal wall proximate the cardiac conduction system and prior to perforation into the LV chamber based on the cardiac conduction system capture, the injury of current, and the impedance comprises determining that the implantable electrode is implanted in the interventricular septal wall proximate the left bundle branch (LBB) for LBB pacing or LBB area pacing.
Example Ex22: The system or method as in any one of Examples Ex19-21, wherein issuing a first notification in response to determining that the implantable electrode is implanted in the interventricular septal wall proximate the cardiac conduction system and prior to perforation into the LV chamber based on the injury of current comprises determining that the implantable electrode is implanted in the interventricular septal wall proximate the cardiac conduction system and prior to perforation into the LV chamber in response to determining a decrease in monitored injury of current while the implantable electrode is moved through the ventricular septum towards the LV chamber during implantation of the implantable electrode.
Example Ex23: The system or method as in Example Ex22, determining the decrease in monitored injury of current comprises determining the decrease in monitored injury of current (IOC) by an IOC implantation change value, wherein the IOC implantation change value is greater than 0 mV.
Example Ex24: The system or method as in any one of Examples Ex19-23, wherein a cardiac conduction system capture threshold is a minimum amount of power utilized to deliver the cardiac conduction system pacing while achieving capture of the cardiac conduction system, wherein issuing a first notification in response to determining that the implantable electrode is implanted in the interventricular septal wall proximate the cardiac conduction system and prior to perforation into the LV chamber based on the monitored cardiac conduction system capture comprises determining that the implantable electrode is implanted in the interventricular septal wall proximate the cardiac conduction system and prior to perforation into the LV chamber in response to determination of an increase in the cardiac conduction system capture threshold while the implantable electrode is moved through the ventricular septum towards the LV chamber during implantation of the implantable electrode.
Example Ex25: The system or method as in Example Ex24, wherein determining the increase in the cardiac conduction system capture threshold comprises determination that the cardiac conduction system capture threshold has increased by an implantation threshold, wherein the implantation threshold is greater than or equal to 1.0 Volt.
Example Ex26: The system or method as in any one of Examples Ex 19-25, wherein issuing a first notification in response to determining that the implantable electrode is implanted in the interventricular septal wall proximate the cardiac conduction system and prior to perforation into the LV chamber based on the impedance comprises determining that the implantable electrode is implanted in the interventricular septal wall proximate the cardiac conduction system and prior to perforation into the LV chamber in response to determining a decrease in monitored impedance while the implantable electrode is moved through the ventricular septum towards the LV chamber during implantation of the implantable electrode.
Example Ex27: The system or method as in Example Ex26, wherein determining the decrease in monitored impedance comprises determining the decrease in monitored impedance by an impedance implantation change value, wherein the impedance implantation change value is greater than or equal to 100 ohms.
Example Ex28: The system as in Example Ex19 or the method as in Example Ex20, wherein the computing apparatus is further configured to execute or the method further comprises, during implantation of the implantable electrode: issuing a second notification in response to determining that the implantable electrode perforated into the LV chamber based on the cardiac conduction system capture threshold, the injury of current, and the impedance.
Example Ex29: The system or method as in Example Ex28, wherein issuing a second notification in response to determining that the implantable electrode has perforated into the LV chamber based on the injury of current comprises determining that the implantable electrode has perforated into the LV chamber in response to determining a decrease in monitored injury of current while the implantable electrode is moved through the ventricular septum from an implantation position proximate the cardiac conduction system into the LV chamber during implantation of the implantable electrode.
Example Ex30: The system or method as in Example Ex29, wherein determining the decrease in monitored injury of current comprises determining the decrease in monitored injury of current by an IOC perforation change value, wherein the IOC perforation change value is greater than or equal to 1 mV.
Example Ex31: The system or method as in Example Ex28, wherein a cardiac conduction system capture threshold is a minimum amount of power utilized to deliver the cardiac conduction system pacing while achieving capture of the cardiac conduction system, wherein issuing a second notification in response to determining that the implantable electrode has perforated into the LV chamber based on the monitored cardiac conduction system capture comprises determining that the implantable electrode has perforated into the LV chamber in response to determining an increase in the cardiac conduction system capture threshold while the implantable electrode is moved through the ventricular septum from an implantation position proximate the cardiac conduction system into the LV chamber during implantation of the implantable electrode.
Example Ex32: The system or method as in Example Ex31, wherein determining the increase in the cardiac conduction system capture threshold comprises determination that the cardiac conduction system capture threshold has increased by a perforation threshold, wherein the perforation threshold is greater than or equal to 1.0 Volt.
Example Ex33: The system or method as in Example Ex28, wherein issuing a second notification in response to determining that the implantable electrode has perforated into the LV chamber based on the impedance comprises determining that the implantable electrode has perforated into the LV chamber in response to determining a decrease in monitored impedance while the implantable electrode is moved through the ventricular septum from an implantation position proximate the cardiac conduction system into the LV chamber during implantation of the implantable electrode.
Example Ex34: The system or method as in Example Ex33, wherein determining the decrease in monitored impedance comprises determining the decrease in monitored impedance by an impedance perforation change value, wherein the impedance perforation change value is greater than or equal to 100 ohms.
Example Ex35: The system as in Example Ex19 or the method as in Example Ex20, wherein the computing apparatus is further configured to execute or the method further comprises: displaying the first notification during implantation of the implantable electrode using a display, wherein the computing apparatus is operatively couplable to the display.
Example Ex36: The system or method as in Example Ex28, further comprising a display, wherein the computing apparatus is further configured to execute or the method further comprises: displaying the first notification during implantation of the implantable electrode using a display; and displaying the second notification during implantation of the implantable electrode using the display, wherein the computing apparatus is operatively couplable to the display.

This disclosure has been provided with reference to illustrative embodiments and examples and is not meant to be construed in a limiting sense. As described previously, one skilled in the art will recognize that other various illustrative applications may use the techniques as described herein to take advantage of the beneficial characteristics of the devices and methods described herein. Various modifications of the illustrative embodiments and examples will be apparent upon reference to this description.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

All references and publications cited herein are expressly incorporated herein by reference in their entirety for all purposes, except to the extent any aspect directly contradicts this disclosure.

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

The recitation of numerical ranges by endpoints includes all numbers subsumed within that range (e.g., 1 to 5 includes 1, 1.5, 2, 2.75, 3, 3.80, 4, and 5) and any range within that range. Herein, the terms “up to” or “no greater than” a number (e.g., up to 50) includes the number (e.g., 50), and the term “no less than” a number (e.g., no less than 5) includes the number (e.g., 5).

The terms “coupled” or “connected” refer to elements being attached to each other either directly (in direct contact with each other) or indirectly (having one or more elements between and attaching the two elements). Either term may be modified by “operatively” and “operably,” which may be used interchangeably, to describe that the coupling or connection is configured to allow the components to interact to carry out at least some functionality (for example, a mobile user device may be operatively coupled to a cellular network transmit data to or receive data therefrom).

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure.

Furthermore, the particular features, configurations, compositions, or characteristics may be combined in any suitable manner in one or more embodiments.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

As used herein, “have,” “having,” “include,” “including,” “comprise,” “comprising” or the like are used in their open-ended sense, and generally mean “including, but not limited to.” It will be understood that “consisting essentially of,” “consisting of,” and the like are subsumed in “comprising,” and the like.

The term “and/or” means one or all of the listed elements or a combination of at least two of the listed elements.

The phrases “at least one of,” “comprises at least one of,” and “one or more of” followed by a list refers to any one of the items in the list and any combination of two or more items in the list.

1. A system for use in assisting implantation of an implantable electrode, the system comprising:
an implantable electrode configured to deliver cardiac conduction system pacing proximate a portion of a patient's cardiac conduction system;
an external electrode configured to at least sense electrical activity of the patient's heart; and
a computing apparatus comprising processing circuitry, the computing apparatus operably coupled to the implantable electrode and the external electrode, wherein the computing apparatus is configured to, during implantation of the implantable electrode:
monitor internal electrical activity using the implantable electrode during implantation of the implantable electrode,
monitor external electrical activity using the external electrode during implantation of the implantable electrode,
determine cardiac conduction system capture based on at least one of the internal and external monitored electrical activity during implantation of the implantable electrode,
determine injury of current based on at least one of the monitored internal and external electrical activity during implantation of the implantable electrode,
determine impedance based on at least one of the monitored internal and external electrical activity during implantation of the implantable electrode, and
issue a first notification in response to determining that the implantable electrode is implanted in the interventricular septal wall proximate the cardiac conduction system and prior to perforation into the left ventricular (LV) chamber based on at least one of the cardiac conduction system capture, the injury of current, and the impedance.

2. The system as in claim 1, wherein issuing a first notification in response to determining that the implantable electrode is implanted in the interventricular septal wall proximate the cardiac conduction system and prior to perforation into the LV chamber based on at least one of the cardiac conduction system capture, the injury of current, and the impedance comprises determining that the implantable electrode is implanted in the interventricular septal wall proximate the left bundle branch (LBB) for LBB pacing or LBB area pacing.

3. The system of claim 1, wherein issuing a first notification in response to determining that the implantable electrode is implanted in the interventricular septal wall proximate the cardiac conduction system and prior to perforation into the LV chamber based on the injury of current comprises determining that the implantable electrode is implanted in the interventricular septal wall proximate the cardiac conduction system and prior to perforation into the LV chamber in response to determining a decrease in monitored injury of current while the implantable electrode is moved through the ventricular septum towards the LV chamber during implantation of the implantable electrode.

4. The system as in claim 3, wherein determining the decrease in monitored injury of current comprises determining the decrease in monitored injury of current (IOC) by an IOC implantation change value, wherein the IOC implantation change value is greater than 0 mV.

5. The system as in claim 1, wherein a cardiac conduction system capture threshold is a minimum amount of power utilized to deliver the cardiac conduction system pacing while achieving capture of the cardiac conduction system,
wherein issuing a first notification in response to determining that the implantable electrode is implanted in the interventricular septal wall proximate the cardiac conduction system and prior to perforation into the LV chamber based on the monitored cardiac conduction system capture comprises determining that the implantable electrode is implanted in the interventricular septal wall proximate the cardiac conduction system and prior to perforation into the LV chamber in response to determination of an increase in the cardiac conduction system capture threshold while the implantable electrode is moved through the ventricular septum towards the LV chamber during implantation of the implantable electrode.

6. The system as in claim 5, wherein determining the increase in the cardiac conduction system capture threshold comprises determination that the cardiac conduction system capture threshold has increased by an implantation threshold, wherein the implantation threshold is greater than or equal to 1.0 Volt.

7. The system as in claim 1, wherein issuing a first notification in response to determining that the implantable electrode is implanted in the interventricular septal wall proximate the cardiac conduction system and prior to perforation into the LV chamber based on the impedance comprises determining that the implantable electrode is implanted in the interventricular septal wall proximate the cardiac conduction system and prior to perforation into the LV chamber in response to determining a decrease in monitored impedance while the implantable electrode is moved through the ventricular septum towards the LV chamber during implantation of the implantable electrode.

8. The system as in claim 7, wherein determining the decrease in monitored impedance comprises determining the decrease in monitored impedance by an impedance implantation change value, wherein the impedance implantation change value is greater than or equal to 100 ohms.

9. The system as in claim 1, wherein the computing apparatus is further configured to execute during implantation of the implantable electrode:
issuing a second notification in response to determining that the implantable electrode perforated into the LV chamber based on at least one of the cardiac conduction system capture threshold, the injury of current, and the impedance.

10. The system as in claim 9, wherein issuing a second notification in response to determining that the implantable electrode has perforated into the LV chamber based on the injury of current comprises determining that the implantable electrode has perforated into the LV chamber in response to determining a decrease in monitored injury of current while the implantable electrode is moved through the ventricular septum from an implantation position proximate the cardiac conduction system into the LV chamber during implantation of the implantable electrode.

11. The system as in claim 10, wherein determining the decrease in monitored injury of current comprises determining the decrease in monitored injury of current by an IOC perforation change value, wherein the IOC perforation change value is greater than or equal to 1 mV.

12. The system as in claim 9, wherein a cardiac conduction system capture threshold is a minimum amount of power utilized to deliver the cardiac conduction system pacing while achieving capture of the cardiac conduction system,
wherein issuing a second notification in response to determining that the implantable electrode has perforated into the LV chamber based on the monitored cardiac conduction system capture comprises determining that the implantable electrode has perforated into the LV chamber in response to determining an increase in the cardiac conduction system capture threshold while the implantable electrode is moved through the ventricular septum from an implantation position proximate the cardiac conduction system into the LV chamber during implantation of the implantable electrode.

13. The system as in claim 12, wherein determining the increase in the cardiac conduction system capture threshold comprises determination that the cardiac conduction system capture threshold has increased by a perforation threshold, wherein the perforation threshold is greater than or equal to 1.0 Volt.

14. The system as in claim 9, wherein issuing a second notification in response to determining that the implantable electrode has perforated into the LV chamber based on the impedance comprises determining that the implantable electrode has perforated into the LV chamber in response to determining a decrease in monitored impedance while the implantable electrode is moved through the ventricular septum from an implantation position proximate the cardiac conduction system into the LV chamber during implantation of the implantable electrode.

15. The system as in claim 14, wherein determining the decrease in monitored impedance comprises determining the decrease in monitored impedance by an impedance perforation change value, wherein the impedance perforation change value is greater than or equal to 100 ohms.

16. A method to assist in implanting an implantable electrode comprising an implantable electrode proximate a patient's cardiac conduction system, the method comprising, during implantation of the implantable electrode:
monitoring internal electrical activity using the implantable electrode during implantation of the implantable electrode,
monitoring external electrical activity using an external electrode during implantation of the implantable electrode,
determining cardiac conduction system capture based on at least one of the internal and external monitored electrical activity during implantation of the implantable electrode,
determining injury of current based on at least one of the monitored internal and external electrical activity during implantation of the implantable electrode,
determining impedance based on at least one of the monitored internal and external electrical activity during implantation of the implantable electrode, and
issuing a first notification in response to determining that the implantable electrode is implanted in the interventricular septal wall proximate the cardiac conduction system and prior to perforation into the left ventricular (LV) chamber based on at least one of the cardiac conduction system capture, the injury of current, and the impedance.

17. The method as in claim 16, wherein issuing a first notification in response to determining that the implantable electrode is implanted in the interventricular septal wall proximate the cardiac conduction system and prior to perforation into the LV chamber based on at least one of the cardiac conduction system capture, the injury of current, and the impedance comprises determining that the implantable electrode is implanted in the interventricular septal wall proximate the left bundle branch (LBB) for LBB pacing or LBB area pacing.

18. The method as in claim 16, wherein issuing a first notification in response to determining that the implantable electrode is implanted in the interventricular septal wall proximate the cardiac conduction system and prior to perforation into the LV chamber based on the injury of current comprises determining that the implantable electrode is implanted in the interventricular septal wall proximate the cardiac conduction system and prior to perforation into the LV chamber in response to determining a decrease in monitored injury of current while the implantable electrode is moved through the ventricular septum towards the LV chamber during implantation of the implantable electrode.

19. The method as in claim 16, wherein a cardiac conduction system capture threshold is a minimum amount of power utilized to deliver the cardiac conduction system pacing while achieving capture of the cardiac conduction system,
wherein issuing a first notification in response to determining that the implantable electrode is implanted in the interventricular septal wall proximate the cardiac conduction system and prior to perforation into the LV chamber based on the monitored cardiac conduction system capture comprises determining that the implantable electrode is implanted in the interventricular septal wall proximate the cardiac conduction system and prior to perforation into the LV chamber in response to determination of an increase in the cardiac conduction system capture threshold while the implantable electrode is moved through the ventricular septum towards the LV chamber during implantation of the implantable electrode.

20. The method as in claim 16, wherein issuing a first notification in response to determining that the implantable electrode is implanted in the interventricular septal wall proximate the cardiac conduction system and prior to perforation into the LV chamber based on the impedance comprises determining that the implantable electrode is implanted in the interventricular septal wall proximate the cardiac conduction system and prior to perforation into the LV chamber in response to determining a decrease in monitored impedance while the implantable electrode is moved through the ventricular septum towards the LV chamber during implantation of the implantable electrode.

21. The method as in claim 16, wherein the method further comprises, during implantation of the implantable electrode:
issuing a second notification in response to determining that the implantable electrode perforated into the LV chamber based on at least one of the cardiac conduction system capture threshold, the injury of current, and the impedance.

22. The method as in claim 21, wherein issuing a second notification in response to determining that the implantable electrode has perforated into the LV chamber based on the injury of current comprises determining that the implantable electrode has perforated into the LV chamber in response to determining a decrease in monitored injury of current while the implantable electrode is moved through the ventricular septum from an implantation position proximate the cardiac conduction system into the LV chamber during implantation of the implantable electrode.

23. The method as in claim 21, wherein a cardiac conduction system capture threshold is a minimum amount of power utilized to deliver the cardiac conduction system pacing while achieving capture of the cardiac conduction system,
wherein issuing a second notification in response to determining that the implantable electrode has perforated into the LV chamber based on the monitored cardiac conduction system capture comprises determining that the implantable electrode has perforated into the LV chamber in response to determining an increase in the cardiac conduction system capture threshold while the implantable electrode is moved through the ventricular septum from an implantation position proximate the cardiac conduction system into the LV chamber during implantation of the implantable electrode.

24. The method as in claim 21, wherein issuing a second notification in response to determining that the implantable electrode has perforated into the LV chamber based on the impedance comprises determining that the implantable electrode has perforated into the LV chamber in response to determining a decrease in monitored impedance while the implantable electrode is moved through the ventricular septum from an implantation position proximate the cardiac conduction system into the LV chamber during implantation of the implantable electrode.

25. A system for use in assisting implantation of an implantable electrode, the system comprising:
an implantable electrode configured to deliver cardiac conduction system pacing proximate a portion of a patient's cardiac conduction system; and
a computing apparatus comprising processing circuitry, the computing apparatus operably coupled to the implantable electrode, wherein the computing apparatus is configured to, during implantation of the implantable electrode:
monitor internal electrical activity using the implantable electrode during implantation of the implantable electrode,
determine cardiac conduction system capture, injury of current, and impedance based on the monitored internal electrical activity during implantation of the implantable electrode, and
issue a first notification in response to determining that the implantable electrode is implanted in the interventricular septal wall proximate the cardiac conduction system and prior to perforation into the left ventricular (LV) chamber based on at least one of the determined cardiac conduction system capture, the injury of current, and the impedance.
